SEQUENCE LISTING (4 sequences)

SEQ ID NO: 1 - protein, 44 aa, Artificial sequence
  Modified GRF peptide
  MISC_FEATURE (1)..(1): Tyr residue is linked to an hexenoyl-trans-3 moiety
  MOD_RES (44)..(44): AMIDATION
YADAIFTNSYRKVLGQLSARKLLQDIMSRQQGESNQERGARARL

SEQ ID NO: 2 - protein, 44 aa, Artificial sequence
  GRF peptide
  VARIANT (1)..(1): Xaa = Tyr or His
  VARIANT (2)..(2): Xaa = Val or Ala
  VARIANT (8)..(8): Xaa = Asn or Ser
  VARIANT (13)..(13): Xaa = Val or Ile
  VARIANT (15)..(15): Xaa = Ala or Gly
  VARIANT (18)..(18): Xaa = Ser or Tyr
  VARIANT (24)..(24): Xaa = Gln or His
  VARIANT (25)..(25): Xaa = Asp or Glu
  VARIANT (27)..(27): Xaa = Met or Ile or Nle
  VARIANT (28)..(28): Xaa = Ser or Asn
  VARIANT (30)..(30): Xaa = any amino acid or is absent
  VARIANT (31)..(31): Xaa = any amino acid or is absent
  misc_feature (32)..(32): Xaa can be any naturally occurring amino acid
  VARIANT (33)..(33): Xaa = any amino acid or is absent
  VARIANT (34)..(34): Xaa = any amino acid or is absent
  VARIANT (35)..(35): Xaa = any amino acid or is absent
  VARIANT (36)..(36): Xaa = any amino acid or is absent
  VARIANT (37)..(37): Xaa = any amino acid or is absent
  VARIANT (38)..(38): Xaa = any amino acid or is absent
  VARIANT (39)..(39): Xaa = any amino acid or is absent
  VARIANT (40)..(40): Xaa = any amino acid or is absent
  VARIANT (41)..(41): Xaa = any amino acid or is absent
  VARIANT (42)..(42): Xaa = any amino acid or is absent
  VARIANT (43)..(43): Xaa = any amino acid or is absent
  VARIANT (44)..(44): Xaa = any amino acid or is absent
XXDAIFYXSYRKXLXQLXARKLLXXIXXRXXXXXXXXXXXXXXX

SEQ ID NO: 3 - protein, 15 aa, Artificial sequence
  Amino acid sequence corresponding to positions 30 to 44 of the human GRF
QQGESNQERGARARL

SEQ ID NO: 4 - protein, 44 aa, Homo sapiens; identical to SEQ ID NO: 1

(19) United States
(12) Patent Application Publication
(10) Pub. No.: US 2022/0378880 A1
(43) Pub. Date: Dec. 1, 2022

(54) PHARMACEUTICAL COMPOSITIONS OF GHRH ANALOGS AND USES THEREOF

(71) Applicant: Theratechnologies Inc., Montréal (CA)

(72) Inventors: Christian Marsolais, Ville Mont-Royal (CA); Kirill Shingel, Brossard (CA); Diane Potvin, Montreal (CA)

(21) Appl. No.: 17/846,990

(22) Filed: Jun. 22, 2022

Related U.S. Application Data
(63) Continuation of application No. 17/358,734, filed on Jun. 25, 2021.
Application No. 17/358,734 is a continuation of application No. 16/361,990, filed on Mar. 22, 2019, now abandoned.

(51) Int. Cl. A61K 38/25; A61K 9/00; A61K 47/40; A61K 47/26; A61K 47/18

(52) U.S. Cl. CPC A61K 38/25; A61K 9/0019; A61K 47/40; A61K 47/26; A61K 47/183

(57) ABSTRACT

A pharmaceutical composition comprising a GHRH molecule or a pharmaceutically acceptable salt thereof is described, as well as uses thereof and a kit for preparing such a pharmaceutical composition. In an embodiment, GHRH molecule or pharmaceutically acceptable salt thereof is trans-3-hexenoyl-GHRH₍₁₋₄₄₎-NH₂ or a pharmaceutically acceptable salt thereof. In an embodiment, a pharmaceutical composition comprising about 1.3 to about 1.5 mg of a GHRH molecule such as trans-3-hexenoyl-GHRH₍₁₋₄₄₎-NH₂ at a concentration of about 3.5 mg/mL or more, as well as uses thereof and a kit for preparing such a pharmaceutical composition, are described. Uses of such a pharmaceutical composition to obtain plasmatic levels of e.g., trans-3-hexenoyl-GHRH₁₋₄₄)-NH₂ that are bioequivalent to administration of 2 mg of trans-3-hexenoyl-GHRH₍₁₋₄₄₎-NH₂ at a concentration of 1 mg/mL in a subject are also described.

### CROSS-REFERENCE TO RELATED APPLICATIONS

This application is a continuation and claims priority of pending U.S. application Ser. No. 17/358,734, filed Jun. 25, 2021, which is a continuation of U.S. application Ser. No. 16/361,990, filed Mar. 22, 2019, now abandoned, the contents of which are incorporated herein by reference in their entirety.

### SEQUENCE LISTING

This application contains a Sequence Listing that has been submitted electronically as an ASCII text file named “Sequence_Listing.txt.” The ASCII text file, created on Jun. 22, 2022, is 5 kilobytes in size. The material in the ASCII text file is hereby incorporated by reference in its entirety.

### TECHNICAL FIELD

The present disclosure generally relates to the field of growth hormone (GH) secretagogues, and more specifically to formulations of Growth Hormone-Releasing Hormone (GHRH) analogs such as tesamorelin and methods of administration thereof.

### BACKGROUND ART

Tesamorelin (trans-3-hexenoyl-GHRH₍₁₋₄₄₎-NH₂, FIG. 1) is a stabilized synthetic peptide analogue of the hypothalamic peptide GHRH indicated for the reduction of excess abdominal fat in HIV-infected patients with lipodystrophy. It mediates its effect by acting on the pituitary somatotroph cells to stimulate the synthesis and pulsatile release of endogenous GH, which is both anabolic and lipolytic. Tesamorelin exerts its therapeutic effects by binding to, and being an agonist of GHRHr on pituitary somatotrophs; the triggered release GH in turn acts on a variety of target cells, including chondrocytes, osteoblasts, myocytes, hepatocytes and adipocytes, resulting in a host of pharmacodynamic effects, which are primarily mediated by insulin-like growth factor 1 (IGF-1) produced in the liver and in peripheral tissues.

The approved daily dosage of tesamorelin for the reduction of excess abdominal fat in HIV-infected patients with lipodystrophy is 2 mg administered by subcutaneous injection of 2 ml of a 1 mg/mL tesamorelin solution into abdominal skin. It is currently supplied to patients in two vials each comprising 1 mg of lyophilized tesamorelin. The patients must resuspend the lyophilized tesamorelin in the first vial with 2.2 mL of sterile water using a syringe with a first mixing needle, collect the prepared tesamorelin solution from the first vial, change the needle, add the prepared tesamorelin solution to the second vial with the second mixing needle, collect the prepared tesamorelin solution from the second vial, replace the second mixing needle with an injection needle, and subcutaneously inject 2 mL of the prepared tesamorelin solution. This relatively complicated process for preparing the injectable tesamorelin solution is not very convenient for patients, and increases the risk of error, contaminations and improper handling of the tesamorelin solution.

There is thus a need for a more simple and convenient method of administration of tesamorelin.

The present description refers to a number of documents, the content of which is herein incorporated by reference in their entirety.

### SUMMARY OF THE INVENTION

The present disclosure generally relates to formulations of Growth Hormone-Releasing Hormone (GHRH) analogs, such as tesamorelin, and methods of administration thereof.

In an aspect, the present disclosure provides a pharmaceutical composition comprising a GHRH molecule or a pharmaceutically acceptable salt thereof (e.g., trans-3-hexenoyl-GHRH₍₁₋₄₄₎-NH₂ or a pharmaceutically acceptable salt thereof) and at least one pharmaceutically acceptable excipient.

In various aspects and embodiments, the present disclosure further provides the following items:
1. A pharmaceutical composition comprising (i) about 1.3 to about 1.6 mg, or about 1.3 to about 1.5, mg of trans-3-hexenoyl-GHRH₍₁₋₄₄₎-NH₂ or a pharmaceutically acceptable salt thereof at a concentration of about 3.5 mg/mL or more; and (ii) at least one pharmaceutically acceptable excipient.
2. The pharmaceutical composition of item 1, comprising about 1.4 mg or 1.5 mg of trans-3-hexenoyl-GHRH₍₁₋₄₄₎-NH₂ or a pharmaceutically acceptable salt thereof.
3. The pharmaceutical composition of item 1, wherein the trans-3-hexenoyl-GHRH₍₁₋₄₄₎-NH₂ or pharmaceutically acceptable salt thereof is at a concentration of about 3.8 to about 10 mg/mL, for example about 4 to about 8 mg/mL, preferably about 3.8 to about 4.2 mg/mL.
4. The pharmaceutical composition of item 3, wherein the trans-3-hexenoyl-GHRH₍₁₋₄₄₎-NH₂ or pharmaceutically acceptable salt thereof is at a concentration of about 4 mg/mL or about 8 mg/m L.
5. The pharmaceutical composition of any one of items 1-4, wherein the at least one pharmaceutically acceptable excipient comprises a diluent.
6. The pharmaceutical composition of item 5, wherein the diluent is sterile water.
7. The pharmaceutical composition of any one of items 1-6, wherein the at least one pharmaceutically acceptable excipient comprises a bulking agent.
8. The pharmaceutical composition of item 7, wherein the bulking agent is mannitol.
9. The pharmaceutical composition of any one of items 1-8, wherein the at least one pharmaceutically acceptable excipient comprises a stabilizer.
10. The pharmaceutical composition of item 9, wherein the stabilizer is sucrose.
11. The pharmaceutical composition of any one of items 1-10, wherein the at least one pharmaceutically acceptable excipient comprises a surfactant.
12. The pharmaceutical composition of item 11, wherein the surfactant is polysorbate 20.
13. The pharmaceutical composition of any one of items 1-12, wherein the at least one pharmaceutically acceptable excipient comprises a buffering agent.
14. The pharmaceutical composition of item 13, wherein the buffering agent is histidine.
15. The pharmaceutical composition of any one of items 1-14, wherein the at least one pharmaceutically acceptable excipient comprises a cyclodextrin.
16. The pharmaceutical composition of claim 15, wherein the cyclodextrin is a β-cyclodextrin.
17. The pharmaceutical composition of any one of items 1-16, wherein the pharmaceutically acceptable salt of trans-3-hexenoyl-GHRH₍₁₋₄₄₎-NH₂ is an acetate salt.
18. A method of administering trans-3-hexenoyl-GHRH₍₁₋₄₄₎-NH₂ or a pharmaceutically acceptable salt thereof to a subject to obtain plasmatic levels of trans-3-hexenoyl-GHRH₍₁₋₄₄₎-NH₂ or a pharmaceutically acceptable salt thereof that are bioequivalent to administration of 2 mg of trans-3-hexenoyl-GHRH₍₁₋₄₄₎-NH₂ at a concentration of 1 mg/mL, the method comprising administering to the subject about 1.3 to about 1.6 mg or about 1.3 to about 1.5 mg of trans-3-hexenoyl-GHRH₍₁₋₄₄₎-NH₂ or a pharmaceutically acceptable salt thereof at a concentration of about 3.5 mg/mL or more.
19. The method of item 18, comprising administering about 1.4 mg or 1.5 mg of trans-3-hexenoyl-GHRH₍₁₋₄₄₎-NH₂ or a pharmaceutically acceptable salt thereof.
20. The method of item 18 or 19, wherein the trans-3-hexenoyl-GHRH₍₁₋₄₄₎-NH₂ or pharmaceutically acceptable salt thereof is at a concentration of about 3.8 to about 10 mg/mL, for example about 4 to about 8 mg/mL, preferably about 3.8 to about 4.2 mg/mL.
21. The method of any one of items 18-20, wherein the pharmaceutically acceptable salt of trans-3-hexenoyl-GHRH₍₁₋₄₄₎-NH₂ is an acetate salt.
22. The method of any one of items 18-21, wherein the subject suffers from HIV-associated lipodystrophy.
23. The method of any one of items 18-22, wherein the trans-3-hexenoyl-GHRH₍₁₋₄₄₎-NH₂ or pharmaceutically acceptable salt thereof is administered by subcutaneous injection.
24. The method of any one of items 18-23, further comprising
   resuspending lyophilized trans-3-hexenoyl-GHRH₍₁₋₄₄₎-NH₂ or pharmaceutically acceptable salt thereof in a suitable amount of a pharmaceutically acceptable diluent to obtain a trans-3-hexenoyl-GHRH₍₁₋₄₄₎-NH₂ or trans-3-hexenoyl-GHRH₍₁₋₄₄₎-NH₂ salt solution at a concentration of about 3.5 mg/mL or more;
   wherein a suitable volume of the trans-3-hexenoyl-GHRH₍₁₋₄₄₎-NH₂ or trans-3-hexenoyl-GHRH₍₁₋₄₄₎-NH₂ salt solution is administered so that about 1.3 to about 1.6 mg or about 1.3 to about 1.5 mg of trans-3-hexenoyl-GHRH₁₋₄₄)-NH₂ or pharmaceutically acceptable salt thereof is administered to the subject.
      25. A method of administering trans-3-hexenoyl-GHRH₍₁₋₄₄₎-NH₂ or a pharmaceutically acceptable salt thereof to a human subject to obtain:
   (i) a maximum plasmatic concentration (Cₘₐₓ) of trans-3-hexenoyl-GHRH₍₁₋₄₄₎-NH₂ or a pharmaceutically acceptable salt thereof of about 1500 to about 4500 pg/mL in the subject;
   (ii) an area under the plasma concentration time curve extrapolated to infinity (AUC_{0-∞}) of trans-3-hexenoyl-GHRH₍₁₋₄₄₎-NH₂ or a pharmaceutically acceptable salt thereof of about 300 to about 1400 p·gh/mL in the subject;
   (iii) a mean plasma concentration-time profile substantially similar to that set forth in FIGS. 2 and/or 3; or
   (iv) any combination of (i) to (iii);
      the method comprising administering to the subject about 1.3 to about 1.6 mg or about 1.3 to about 1.5 mg of trans-3-hexenoyl-GHRH₍₁₋₄₄₎-NH₂ or a pharmaceutically acceptable salt thereof at a concentration of about 3.5 mg/mL or more.
26. The method of item 25, comprising administering about 1.4 mg or 1.5 mg of trans-3-hexenoyl-GHRH₍₁₋₄₄₎-NH₂ or a pharmaceutically acceptable salt thereof.
27. The method of item 25 or 26, wherein the trans-3-hexenoyl-GHRH₍₁₋₄₄₎-NH₂ or pharmaceutically acceptable salt thereof is at a concentration of about 3.8 to about 10 mg/mL, for example about 4 to about 8 mg/mL, preferably about 3.8 to about 4.2 mg/mL.
28. The method of any one of items 25-27, wherein the pharmaceutically acceptable salt of trans hexenoyl-GHRH₍₁₋₄₄₎-NH₂ is an acetate salt.
29. The method of any one of items 25-28, wherein the subject suffers from HIV-associated lipodystrophy.
30. The method of any one of items 25-29, wherein the trans-3-hexenoyl-GHRH₍₁₋₄₄₎-NH₂ or pharmaceutically acceptable salt thereof is administered by subcutaneous injection.
31. The method of any one of items 25-30, further comprising resuspending lyophilized trans-3-hexenoyl-GHRH₍₁₋₄₄₎-NH₂ or pharmaceutically acceptable salt thereof in a suitable amount of a pharmaceutically acceptable diluent to obtain a trans-3-hexenoyl-GHRH₍₁₋₄₄₎-NH₂ or trans-3-hexenoyl-GHRH₍₁₋₄₄₎-NH₂ salt solution at a concentration of about 3.5 mg/mL or more;
   wherein a suitable volume of the trans-3-hexenoyl-GHRH₍₁₋₄₄₎-NH₂ or trans-3-hexenoyl-GHRH₍₁₋₄₄₎-NH₂ salt solution is administered so that about 1.3 to about 1.6 mg or about 1.3 to about 1.5 mg of trans-3-hexenoyl-GHRH-₍₁₋₄₄₎-NH₂ or pharmaceutically acceptable salt thereof is administered to the subject.
32. A kit comprising:
   (a) a first container comprising at least about 1.3 to about 1.6 mg or about 1.3 to about 1.5 mg of lyophilized trans-3-hexenoyl-GHRH₍₁₋₄₄₎-NH₂ or a pharmaceutically acceptable salt thereof;
   (b) a second container comprising a pharmaceutically acceptable diluent; and
   (c) instructions setting forth the method of item 24.
33. The kit of item 32, further comprising at least one syringe.
34. A pharmaceutical composition comprising trans-3-hexenoyl-GHRH₍₁₋₄₄₎-NH₂ or a pharmaceutically acceptable salt thereof for use in producing plasmatic levels of trans-3-hexenoyl-GHRH₍₁₋₄₄₎-NH₂ or a pharmaceutically acceptable salt thereof in a subject that are bioequivalent to administration of 2 mg of trans-3-hexenoyl-GHRH₍₁₋₄₄₎-NH₂ at a concentration of mg/mL, wherein the pharmaceutical composition is for administration of about 1.3 to about 1.6 mg or about 1.3 to about 1.5 mg of trans-3-hexenoyl-GHRH₍₁₋₄₄₎-NH₂ or a pharmaceutically acceptable salt thereof at a concentration of about 3.5 mg/mL or more, to the subject.
35. A pharmaceutical composition comprising trans-3-hexenoyl-GHRH₍₁₋₄₄₎-NH₂ or a pharmaceutically acceptable salt thereof for use in administration to a human subject for producing:
   (i) a maximum plasmatic concentration (Cₘₐₓ) of trans-3-hexenoyl-GHRH₍₁₋₄₄₎-NH₂ or a pharmaceutically acceptable salt thereof of about 1500 to about 4500 pg/mL in the subject;
   (ii) an area under the plasma concentration time curve extrapolated to infinity (AUC_{0-∞}) of trans-3-hexenoyl-GHRH₍₁₋₄₄₎-NH₂ or a pharmaceutically acceptable salt thereof of about 300 to about 1400 pg h/mL in the subject;
   (iii) a mean plasma concentration-time profile substantially similar to that set forth in FIG. 5 and/or FIG. 6; or
   (iv) any combination of (i) to (iii);
wherein the pharmaceutical composition is for administration of about 1.3 to about 1.6 mg or about 1.3 to about 1.5 mg of trans-3-hexenoyl-GHRH₍₁₋₄₄₎-NH₂ or a pharmaceutically acceptable salt thereof at a concentration of about 3.5 mg/mL or more, to the subject.
36. The pharmaceutical composition for use of item 34 or 35, wherein the pharmaceutical composition is for administration of about 1.4 or 1.5 mg of trans-3-hexenoyl-GHRH₍₁₋₄₄₎-NH₂ or a pharmaceutically acceptable salt thereof.
37. The pharmaceutical composition for use of any one of items 34 to 36, wherein the pharmaceutical composition is for administration of the trans-3-hexenoyl-GHRH₍₁₋₄₄₎-NH₂ or pharmaceutically acceptable salt thereof at a concentration of about 3.8 to about 10 mg/mL, for example about 4 to about 8 mg/mL, preferably about 3.8 to about 4.2 mg/mL.
38. The pharmaceutical composition for use of any one of items 34 to 37, wherein the pharmaceutically acceptable salt of trans-3-hexenoyl-GHRH₍₁₋₄₄₎-NH₂ is an acetate salt.
39. The pharmaceutical composition for use of any one of items 34 to 38, wherein the subject suffers from HIV-associated lipodystrophy.
40. The pharmaceutical composition for use of any one of items 34 to 39, wherein the trans-3-hexenoyl-GHRH₍₁₋₄₄₎-NH₂ or pharmaceutically acceptable salt thereof is for administration by subcutaneous injection,
41. The pharmaceutical composition for use of any one of items 34 to 40, further comprising resuspending lyophilized trans-3-hexenoyl-GHRH₍₁₋₄₄₎-NH₂ or pharmaceutically acceptable salt thereof in a suitable amount of a pharmaceutically acceptable diluent to obtain a trans-3-hexenoyl-GHRH₍₁₋₄₄₎NH₂ or trans-3-hexenoyl-GHRH₍₁₋₄₄₎-NH₂ salt solution at a concentration of about 3.5 mg/mL or more; thereby to provide the pharmaceutical composition for administration.
42. Use of a pharmaceutical composition comprising trans-3-hexenoyl-GHRH₍₁₋₄₄₎-NH₂ or a pharmaceutically acceptable salt thereof for producing plasmatic levels of trans-3-hexenoyl-GHRH₍₁₋₄₄₎-NH₂ or a pharmaceutically acceptable salt thereof in a subject that are bioequivalent to administration of 2 mg of trans-3-hexenoyl-GHRH₍₁₋₄₄₎-NH₂ at a concentration of 1 mg/mL, wherein the pharmaceutical composition is for administration of about 1.3 to about 1.6 mg or about 1.3 to about 1.5 mg of trans-3-hexenoyl-GHRH₍₁₋₄₄₎-NH₂ or a pharmaceutically acceptable salt thereof at a concentration of about 3.5 mg/mL or more, to the subject.
43. Use of a pharmaceutical composition comprising trans-3-hexenoyl-GHRH₍₁₋₄₄₎-NH₂ or a pharmaceutically acceptable salt thereof for use in administration to a human subject for producing:
   (i) a maximum plasmatic concentration (Cₘₐₓ) of trans-3-hexenoyl-GHRH₍₁₋₄₄₎-NH₂ or a pharmaceutically acceptable salt thereof of about 1500 to about 4500 pg/mL in the subject;
   (ii) an area under the plasma concentration time curve extrapolated to infinity (AUC_{0-∞}) of trans-3-hexenoyl-GHRH₍₁₋₄₄₎-NH₂ or a pharmaceutically acceptable salt thereof of about 300 to about 1400 pg h/mL in the subject;
   (iii) a mean plasma concentration-time profile substantially similar to that set forth in FIG. 5 and/or FIG. 6; or
   (iv) any combination of (i) to (iii);
wherein the pharmaceutical composition is for administration of about 1.3 to about 1.6 mg or about 1.3 to about 1.5 mg of trans-3-hexenoyl-GHRH₍₁₋₄₄₎-NH₂ or a pharmaceutically acceptable salt thereof at a concentration of about 3.5 mg/mL or more, to the subject.
44. Use of trans-3-hexenoyl-GHRH₍₁₋₄₄₎-NH₂ or a pharmaceutically acceptable salt thereof for the preparation of a pharmaceutical composition for producing plasmatic levels of trans-3-hexenoyl-GHRH₍₁₋₄₄₎-NH₂ or a pharmaceutically acceptable salt thereof in a subject that are bioequivalent to administration of 2 mg of trans-3-hexenoyl-GHRH₍₁₋₄₄₎-NH₂ at a concentration of 1 mg/mL, wherein the pharmaceutical composition is for administration of about 1.3 to about 1.6 mg or about 1.3 to about 1.5 mg of trans-3-hexenoyl-GHRH₍₁₋₄₄₎-NH₂ or a pharmaceutically acceptable salt thereof at a concentration of about 3.5 mg/mL or more, to the subject.
45. Use of trans-3-hexenoyl-GHRH₍₁₋₄₄₎-NH₂ or a pharmaceutically acceptable salt thereof for the preparation of a pharmaceutical composition for administration to a human subject for producing:
   (i) a maximum plasmatic concentration (Cₘₐₓ) of trans-3-hexenoyl-GHRH₍₁₋₄₄₎-NH₂ or a pharmaceutically acceptable salt thereof of about 1500 to about 4500 pg/mL in the subject;
   (ii) an area under the plasma concentration time curve extrapolated to infinity (AUC_{0-∞}) of trans-3-hexenoyl-GHRH₍₁₋₄₄₎-NH₂ or a pharmaceutically acceptable salt thereof of about 300 to about 1400 pg h/mL in the subject;
   (iii) a mean plasma concentration-time profile substantially similar to that set forth in FIG. 5 and/or FIG. 6; or
   (iv) any combination of (i) to (iii);
wherein the pharmaceutical composition is for administration of about 1.3 to about 1.6 mg or about 1.3 to about 1.5 mg of trans-3-hexenoyl-GHRH₍₁₋₄₄₎-NH₂ or a pharmaceutically acceptable salt thereof at a concentration of about 3.5 mg/mL or more, to the subject.
46. The use of any one of items 42 to 45, wherein the pharmaceutical composition is for administration of about 1.4 or about 1.5 mg of trans-3-hexenoyl-GHRH₍₁₋₄₄₎-NH₂ or a pharmaceutically acceptable salt thereof.
47. The use of any one of items 42 to 46, wherein the pharmaceutical composition is for administration of the trans-3-hexenoyl-GHRH₍₁₋₄₄₎-NH₂ or pharmaceutically acceptable salt thereof at a concentration of about 3.8 to about 10 mg/mL, for example about 4 to about 8 mg/mL, preferably about 3.8 to about 4.2 mg/mL.
48. The use of any one of items 42 to 47, wherein the pharmaceutically acceptable salt of trans-3-hexenoyl-GHRH₍₁₋₄₄₎-NH₂ is an acetate salt.
49. The use of any one of items 42 to 48, wherein the subject suffers from HIV-associated lipodystrophy.
50. The use of any one of items 42 to 49, wherein the trans-3-hexenoyl-GHRH₍₁₋₄₄₎-NH₂ or pharmaceutically acceptable salt thereof is for administration by subcutaneous injection.
51. The use of any one of items 42 to 50, further comprising resuspending lyophilized trans-3-hexenoyl-GHRH₍₁₋₄₄₎-NH₂ or pharmaceutically acceptable salt thereof in a suitable amount of a pharmaceutically acceptable diluent to obtain a trans-3-hexenoyl-GHRH₍₁₋₄₄₎-NH₂ or trans-3-hexenoyl-GHRH₍₁₋₄₄₎-NH₂ salt solution at a concentration of about 3.5 mg/mL or more; thereby to provide the formulation for administration.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the structure of tesamorelin (trans-3-hexenoyl-GHRH₍₁₋₄₄₎-NH₂; SEQ ID NO: 1).

FIG. 2 shows a standard curve generated by diluting a stock solution of TH9507 (0.1 mg/mL) in acidified dog plasma diluted 4-fold with an ELISA assay buffer. The standards have a range of 100 ng/mL to 0.1 ng/mL.

FIGS. 3A-3C show a comparison of the pharmacokinetic profile of TH9507 in dogs following administration of formulations #1 and #8.

FIG. 4 is a schematic of the design of the bioequivalence study in humans presented in Example 3.

FIG. 5 shows the linear profile of mean plasma concentrations versus time for tesamorelin. Treatment A: Tesamorelin 4 mg/mL, single subcutaneous dose of 1.4 mg (0.35 mL); Treatment B: Tesamorelin 1 mg/mL, single subcutaneous dose of 2 mg (2.0 mL).

FIG. 6 shows the logarithmic profile of mean plasma concentrations versus time for tesamorelin. Treatments A and B as in FIG. 5.

### DISCLOSURE OF INVENTION

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Similarly, herein a general chemical structure with various substituents and various radicals enumerated for these substituents is intended to serve as a shorthand method of referring individually to each and every molecule obtained by the combination of any of the radicals for any of the substituents. Each individual molecule is incorporated into the specification as if it were individually recited herein. Further, all subsets of molecules within the general chemical structures are also incorporated into the specification as if they were individually recited herein.

All methods described herein can be performed in any suitable order unless otherwise indicated herein or otherwise clearly contradicted by context.

The use of any and all examples, or exemplary language (“e.g.”, “such as”, etc.) provided herein, is intended merely to better illustrate the disclosure and does not pose a limitation on the scope of the disclosure unless otherwise claimed.

No language in the specification should be construed as indicating any non-claimed element as essential to the practice of the disclosure.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Unless otherwise defined, all technical and scientific terms used herein have the same meaning as commonly understood by one of ordinary skill in the art to which this disclosure belongs.

In the studies described herein, the present inventors have shown that tesamorelin formulated at 4 mg/mL is more bioavailable that corresponding tesamorelin formulations at 1 mg/mL. Pharmacokinetic (PK) studies in human subjects have shown that administration of 1.4 mg of tesamorelin formulated at 4 mg/mL is bioequivalent to administration of 2 mg of a 1 mg/mL tesamorelin formulation (e.g., the Egrifta™ formulation), the approved daily dosage of tesamorelin (EGRIFTA®). Thus, it was found that the amount of tesamorelin administered to the subject should be reduced by 30% (i.e. 1.4 mg vs. 2 mg) to obtain bioequivalence in the subjects. This advantageously reduces the volume of administration (0.35 mL vs. 2 mL), and renders the preparation and handling of the formulation more user-friendly as it may be provided in a single vial instead of two, thereby reducing the risk of error and contaminations/infections.

Accordingly, in a first aspect, the present disclosure provides a pharmaceutical composition comprising (i) about 1.3 to about 1.5 mg of a GHRH molecule or a pharmaceutically acceptable salt thereof, preferably trans-3-hexenoyl-GHRH₍₁₋₄₄₎-NH₂ or a pharmaceutically acceptable salt thereof, at a concentration of about 3.5 mg/mL or more; and (ii) at least one pharmaceutically acceptable excipient.

In embodiments, the pharmaceutical composition comprises from about 1.30, 1.31, 1.32, 1.33, 1.34, 1.35, 1.36, 1.37, 1.38 or 1.39 to about 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6 mg of the GHRH molecule, preferably trans-3-hexenoyl-GHRH₍₁₋₄₄₎-NH₂ or a pharmaceutically acceptable salt thereof. In a further embodiment, the pharmaceutical composition comprises from about 1.35 to about 1.45 mg of the GHRH molecule, preferably trans-3-hexenoyl-GHRH₍₁₋₄₄₎-NH₂ or a pharmaceutically acceptable salt thereof. In a further embodiment, the pharmaceutical composition comprises from about 1.36 to about 1.44 mg of the GHRH molecule, preferably trans-3-hexenoyl-GHRH₍₁₋₄₄₎-NH₂ or a pharmaceutically acceptable salt thereof. In a further embodiment, the pharmaceutical composition comprises from about 1.37 to about 1.43 mg of the GHRH molecule, preferably trans-3-hexenoyl-GHRH₍₁₋₄₄₎-NH₂ or a pharmaceutically acceptable salt thereof. In a further embodiment, the pharmaceutical composition comprises from about 1.38 to about 1.42 mg of the GHRH molecule, preferably trans-3-hexenoyl-GHRH₍₁₋₄₄₎-NH₂ or a pharmaceutically acceptable salt thereof. In a further embodiment, the pharmaceutical composition comprises from about 1.39 to about 1.41 mg of the GHRH molecule, preferably trans-3-hexenoyl-GHRH₍₁₋₄₄₎-NH₂ or a pharmaceutically acceptable salt thereof. In a further embodiment, the pharmaceutical composition comprises from about 1.4 mg of the GHRH molecule, preferably trans-3-hexenoyl-GHRH₍₁₋₄₄₎-NH₂ or a pharmaceutically acceptable salt thereof.

In an embodiment, the GHRH molecule, preferably trans-3-hexenoyl-GHRH₍₁₋₄₄₎-NH₂ or a pharmaceutically acceptable salt thereof; is at a concentration of about 12, 10 or 8 mg/mL or less. In embodiments, the GHRH molecule, preferably trans-3-hexenoyl-GHRH₍₁₋₄₄₎-NH₂ or a pharmaceutically acceptable salt thereof, is at a concentration of about 3.5 to about 10, 9, 8, 7, 6 or 5 mg/mL, for example a concentration of about 4 to about 8 mg/mL. In further embodiments, the GHRH molecule, preferably trans-3-hexenoyl-GHRH₍₁₋₄₄₎-NH₂ or a pharmaceutically acceptable salt thereof, is at a concentration of about 3.80, 3.82, 3.84, 3.86, 3.88, 3.9, 3.92, 3.94, 3.95, 3.96, 3.97, 3.98 or 3.99 to about 4.01, 4.02, 4.03, 4.04, 4.05, 4.06, 4.08, 4.1, 4.12, 4.14, 4.16, 4.18 or 4.2 mg/ml in the pharmaceutical composition. In further embodiments, the GHRH molecule, preferably trans-3-hexenoyl-GHRH₍₁₋₄₄₎-NH₂ or a pharmaceutically acceptable salt thereof, is at a concentration of about 7.80, 7.82, 7.84, 7.86, 7.88, 7.9, 7.92, 7.94, 7.95, 7.96, 7.97, 7.98 or 7.99 to about 8.01, 8.02, 8.03, 8.04, 8.05, 8.06, 8.08, 8.1, 8.12, 8.14, 8.16, 8.18 or 8.2 mg/mL in the pharmaceutical composition. In a further embodiment, the GHRH molecule, preferably trans-3-hexenoyl-GHRH₍₁₋₄₄₎-NH₂ or a pharmaceutically acceptable salt thereof, is at a concentration of about 4 mg/mL or about 8 mg/mL.

The term “GHRH molecule” as used in the context of the present disclosure includes, without limitation, human native GHRH₍₁₋₄₄₎ and fragments thereof (e.g., GHRH₍₁₋₄₀₎, GHRH₍₁₋₂₉₎, fragments ranging between 1-29 and the 1-44 sequence), and any other fragments; GHRH from other species and fragments thereof; GHRH variants containing amino acid(s) substitution(s), addition(s) and/or deletion(s); derivatives or analogs of GHRH or fragments or variants thereof having for example an organic group or a moiety coupled to the GHRH amino acid sequence at the N-terminus, the C-terminus or on the side-chain; and pharmaceutically acceptable salts of GHRH (human or from other species), as well as pharmaceutically acceptable salts of GHRH fragments, variants, analogs and derivatives. The GHRH molecules of the present disclosure also encompass the GHRH molecules currently known in the art, including, without limitation, albumin-conjugated GHRH (U.S. Pat. No. 7,268,113); pegylated GHRH peptide (U.S. Pat. Nos. 7,256,258 and 6,528,485); porcine GHRH (1-40) (U.S. Pat. No. 6,551,996); canine GHRH (U.S. patent application no. 2005/0064554); GHRH variants of 1-29 to 1-44 amino acid length (U.S. Pat. Nos. 5,846,936, 5,696,089, 5,756,458 and 5,416,073, and U.S. patent application Nos. 2006/0128615 and 2004/0192593); and Pro⁰-GHRHpeptide and variants thereof (U.S. Pat. No. 5,137,872).

The GHRH analogs include those described in U.S. Pat. Nos. 5,681,379 and 5,939,386, which also describe their method of synthesis. More particularly, these GHRH analogs are defined by the following formula A:

X-GHRH Peptide (A)

wherein the GHRH peptide is a peptide of the following formula B (SEQ ID NO:2):

A1-A2-Asp-Ala-Ile-Phe-Thr-A8-Ser-Tyr-Arg-Lys-A13-Leu-A15-Gln-Leu-A18-Ala-Arg-Lys-Leu-Leu-A24-A25-Ile-A27-A28-Arg-A30-A31-A32-A33-A34-A35-A36-A37-A38-A39-A40-A41-A42-A43-A44-R0 (B)

wherein,
A1 is Tyr or His;
A2 is Val or Ala;
A8 is Asn or Ser;
A13 is Val or Ile;
A15 is Ala or Gly;
A18 is Ser or Tyr;
A24 is Gln or His;
A25 is Asp or Glu;
A27 is Met, Ile or NIe
A28 is Ser or Asn;
A30 is absent or is any amino acid, preferably Gln;
A31 is absent or is any amino acid, preferably Gln;
A32 is absent or is any amino acid, preferably Gly;
A33 is absent or is any amino acid, preferably Glu;
A34 is absent or is any amino acid, preferably Ser;
A35 is absent or is any amino acid, preferably Asn;
A36 is absent or is any amino acid, preferably Gln;
A37 is absent or is any amino acid, preferably Glu;
A38 is absent or is any amino acid, preferably Arg;
A39 is absent or is any amino acid, preferably Gly;
A40 is absent or is any amino acid, preferably Ala;
A41 is absent or is any amino acid, preferably Arg;
A42 is absent or is any amino acid, preferably Ala;
A43 is absent or is any amino acid, preferably Arg;
A44 is absent or is any amino acid, preferably Leu; and
R0 is NH₂ or NH—(CH₂)n-CONH₂, with n=1 to 12.

The group X is a hydrophobic tail anchored via an amide bond to the N-terminus of the peptide and the hydrophobic tail defining a backbone of 5 to 7 atoms. The backbone can be substituted by C₁₋₆ alkyl, C₃₋₆ cycloalkyl, or C₆₋₁₂ aryl and the backbone comprises at least one rigidifying moiety connected to at least two atoms of the backbone. The rigidifying moiety is a double bond, triple bond, saturated or unsaturated C₃₋₉ cycloalkyl, or C₆₋₁₂ aryl.

In an embodiment, group X is:

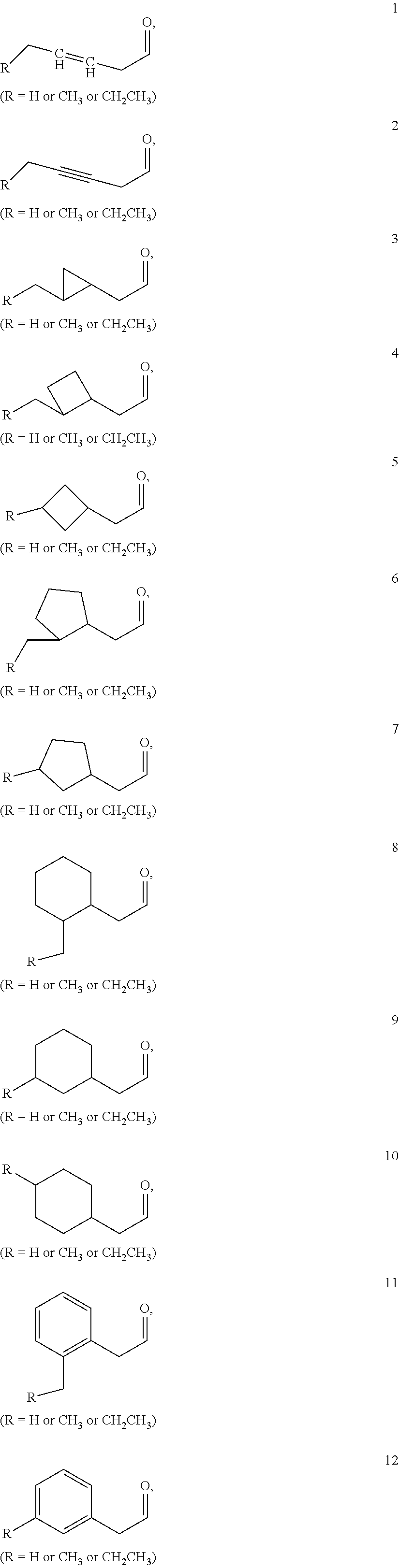

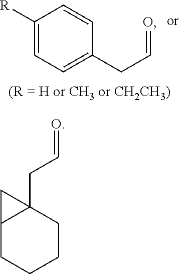

In an embodiment, in formula B, A30-A44 are: (a) absent; (b) an amino acid sequence corresponding to positions 30-44 of a native GHRH peptide (SEQ ID NO: 3), or (c) the amino acid sequence of (b) having a 1-14 amino acid deletion from its C-terminus.

In an embodiment, the GHRH peptide is a polypeptide comprising the amino acid sequence of SEQ ID NO: 4.

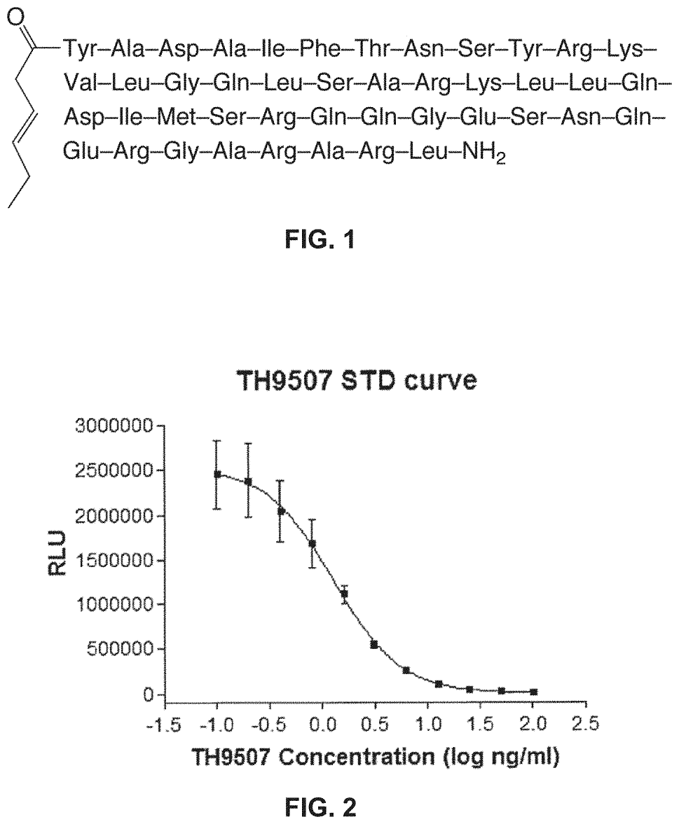

In an embodiment, the GHRH molecule is (hexenoyl trans-3)hGHRH₍₁₋₄₄₎NH₂ (SEQ ID NO: 1) or a pharmaceutically acceptable salt thereof. [trans-3-hexenoyl]hGHRH₍₁₋₄₄₎ amide (also referred to as (hexenoyl trans-3)hGHRH₍₁₋₄₄₎NH₂) is a synthetic human GHRH (hGHRH) analog that comprises the 44-amino acid sequence of hGHRH on which a hexenoyl moiety, a C₆ side chain, has been anchored on the amino-terminal tyrosine residue. The structure of [trans-3-hexenoyl]hGHRH₍₁₋₄₄₎ amide is depicted at FIG. 1.

The term “pharmaceutically acceptable salt” refers to salts of GHRH molecules that are pharmacologically acceptable and substantially non-toxic to the subject to which they are administered. More specifically, these salts retain the biological effectiveness and properties of the GHRH molecule and are formed from suitable non-toxic organic or inorganic acids or bases.

For example, these salts include acid addition salts of GHRH molecules which are sufficiently basic to form such salts. Such acid addition salts include acetates, adipates, alginates, lower alkanesulfonates such as a methanesulfonates, trifluoromethanesulfonatse or ethanesulfonates, arylsulfonates such as a benzenesulfonates, 2-naphthalenesulfonates, or toluenesulfonates (also known as tosylates), ascorbates, aspartates, benzoates, benzenesulfonates, bisulfates, borates, butyrates, citrates, camphorates, camphorsulfonates, cinnamates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydrogen sulphates, 2-hydroxyethanesulfonates, itaconates, lactates, maleates, mandelates, methanesulfonates, nicotinates, nitrates, oxalates, pamoates, pectinates, perchlorates, persulfates, 3-phenylpropionates, phosphates, picrates, pivalates, propionates, salicylates, succinates, sulfates, sulfonates, tartrates, thiocyanates, undecanoates and the like.

Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

Such salts can be formed quite readily by those skilled in the art using standard techniques. Indeed, the chemical modification of a pharmaceutical compound (i.e. drug) into a salt is a technique well known to pharmaceutical chemists, (See, e.g., H. Ansel et. al., Pharmaceutical Dosage Forms and Drug Delivery Systems (6th Ed. 1995) at pp. 196 and 1456-1457). Salts of the GHRH molecules may be formed, for example, by reacting the GHRH molecule with an amount of acid or base, such as an equivalent amount, in a medium such as one in which the salt precipitates or in an aqueous medium followed by lyophilization.

In an embodiment, the pharmaceutically acceptable salt of the GHRH molecule, preferably [trans-3-hexenoyl]hGHRH₍₁₋₄₄₎ amide, is an acetate salt.

The term “pharmaceutically acceptable excipient” as used herein has its normal meaning in the art and is any ingredient that is not an active ingredient (drug) itself. Excipients include for example binders, lubricants, diluents, bulking agents (fillers), thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present composition is not limited in these respects. In certain embodiments, the pharmaceutical composition comprises one or more excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. Therapeutic formulations are prepared using standard methods known in the art by mixing the active ingredient having the desired degree of purity with one or more optional pharmaceutically acceptable carriers, excipients and/or stabilizers. The excipient(s) may be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7th edition, Pharmaceutical Press). In an embodiment, the pharmaceutical composition is an injectable composition. In an embodiment, the pharmaceutical composition comprises one or more excipients for subcutaneous administration/injection.

In an embodiment, the pharmaceutical composition comprises a bulking agent. The term “bulking agent” as used herein refers to a compound used to provide an adequate or desired tonicity of the solution resulting from the reconstitution of the lyophilized formulation. Preferably, the adequate or desired tonicity of the solution is equal to or approximates isotonicity with physiological fluid of the subject to which the solution is administered. For example, one or more sugars may be used as the bulking agent. Sugars, as used herein, include, but are not limited to, monosaccharides, oligosaccharides and polysaccharides. Examples of suitable sugars include, but are not limited to, mannose, sorbose, xylose, maltose, lactose, sucrose, and dextran. Sugar also includes sugar alcohols, such as mannitol, inositol, dulcitol, xylitol and arabitol. Mixtures of sugars may also be used in accordance with the present disclosure. In an embodiment, the bulking agent is mannitol. For example, one or more amino acids, such as glycine, may be used as the bulking agent. The bulking agent is in concentration of about 1% to about 10% (w/w) or about 2% to about 8% (w/w) in the pharmaceutical composition. In an embodiment, the bulking agent is in concentration of about 3 to about 5% (w/w) in the pharmaceutical composition. In a further embodiment, the bulking agent is in concentration of about 4% (w/w) in the pharmaceutical composition.

In an embodiment, the pharmaceutical composition of the present disclosure may further comprise a surfactant. Typical examples of surfactants include sorbitan fatty acid esters such as sorbitan monocaprylate, sorbitan monolaurate, sorbitan monopalmitate; glycerin fatty acid esters such as glycerin monocaprylate, glycerin monomyristate, glycerin monostearate; polyglycerin fatty acid esters such as decaglyceryl monostearate, decaglyceryl distearate, decaglyceryl monolinoleate; polyoxyethylene sorbitan fatty acid esters such as polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate; polyoxyethylene sorbitol fatty acid esters such as polyoxyethylene sorbitol tetrastearate, polyoxyethylene sorbitol tetraoleate; polyoxyethylene glycerin fatty acid esters such as polyoxyethylene glyceryl monostearate; polyethylene glycol fatty acid esters such as polyethylene glycol distearate; polyoxyethylene alkyl ethers such as polyoxyethylene lauryl ether; polyoxyethylene polyoxypropylene alkyl ethers such as polyoxyethylene polyoxypropylene glycol ether, polyoxyethylene polyoxypropylene propyl ether, polyoxyethylene polyoxypropylene cetyl ether; polyoxyethylene alkyl phenyl ethers such as polyoxyethylene nonyl phenyl ether; polyoxyethylene hardened castor oils such as polyoxyethylene castor oil, polyoxyethylene hardened castor oil (polyoxyethylene hydrogenated castor oil); polyoxyethylene beeswax derivatives such as polyoxyethylene sorbitol beeswax; polyoxyethylene lanolin derivatives such as polyoxyethylene lanolin; polyoxyethylene fatty acid amides such as polyoxyethylene stearic acid amide; alkyl sulfates having a C₁₀₋₁₈ alkyl group such as sodium cetyl sulfate, sodium lauryl sulfate, sodium oleyl sulfate; polyoxyethylene alkyl ether sulfates having an average EO mole number of 2-4 and a C₁₀₋₁₈ alkyl group such as sodium polyoxyethylene lauryl sulfate; alkyl sulfosuccinic acid ester salts having a C₈₋₁₈ alkyl group such as sodium laurylsulfosuccinate; lecithin; glycerophospholipids; sphingophospholipids such as sphingomyelin; sucrose fatty acid esters of C₁₂₋₁₈ fatty acids.

In an embodiment, the surfactant the pharmaceutical composition of the present disclosure is a non-ionic surfactant. In a further embodiment, the surfactant the pharmaceutical composition of the present disclosure is polyoxyethylene sorbitan alkyl ester. In yet a further embodiment, the surfactant the pharmaceutical composition of the present disclosure is polysorbate-20 (T20 or Tween-20™).

In another embodiment, the amount of surfactant in the pharmaceutical composition of the present disclosure is about 0.0001% to about 10% (w/w). In a further embodiment, the amount of surfactant in the pharmaceutical composition of the present disclosure is about 0.001% to about 5%, 1% or 0.1% (w/w) or about 0.005% to about 0.05%. In yet a further embodiment, the amount of surfactant in the pharmaceutical composition of the present disclosure is about 0.01% (w/w).

In an embodiment, the pharmaceutical composition of the present disclosure may further comprise one or more stabilizing agents or stabilizers. As used herein, the term “stabilizer” is intended to mean a compound used to stabilize the therapeutic agent against physical, chemical, or biochemical process that would reduce the therapeutic activity of the agent. Suitable stabilizers are non-reducing sugars including, by way of example and without limitation, sucrose (or saccharose) and trehalose; and non-reducing polyols including, by way of example and without limitation, sorbitol, mannitol, maltitol, xylitol, glycol, glycerol and ethylene glycol.

In an embodiment, the pharmaceutical composition of the present disclosure comprises a non-reducing sugar. “Non-reducing sugar” as used herein refers to a sugar that does not contain a hemi-acetal, for example a carbohydrate or sugar characterized by having a glycosidic bond formed between the reducing ends of the sugar units, and not between a reducing end of one sugar unit and a non-reducing end of the other sugar unit. In a further embodiment, the above-mentioned non-reducing sugar is trehalose or sucrose. In a further embodiment, the above-mentioned non-reducing sugar is sucrose. In an embodiment, the non-reducing sugar is in a concentration of about 0.1% to about 5% (w/w) in the pharmaceutical composition of the disclosure. In an embodiment, the non-reducing sugar is in a concentration of about 1% to about 3% (w/w). In a further embodiment, the non-reducing sugar is in a concentration of about 2% (w/w).

In an embodiment, the pharmaceutical composition of the present disclosure comprises a buffering agent, i.e. an agent that maintains the pH of the pharmaceutical composition near a chosen value. Examples of buffering agents include acetate buffers, succinate buffers, citrate buffers, phosphate buffers and histidine buffers. In an embodiment, the buffering agent is a histidine buffer. In an embodiment, the concentration of histidine in the pharmaceutical composition is about 0.01% to about 1%, for example about 0.05% to about 0.5% or about 0.1% to about 0.3%. In a further embodiment, the histidine sugar is in a concentration of about 0.15%.

In an embodiment, the pharmaceutical composition of the present disclosure comprises an oligosaccharide, for example a cyclic oligosaccharide such as a cyclodextrin. The term “cyclodextrin” as used herein refers to a family of cyclic oligosaccharides, comprising a macrocyclic ring of glucopyranoside subunits (5 or more) joined by α-1,4 glycosidic bonds. Examples of cyclodextrins include α-cyclodextrin, β-cyclodextrin and γ-cyclodextrin, which comprise 6, 7 and 8 glucopyranoside subunits, respectively, as well as analogs thereof (e.g., modified cyclodextrins). In an embodiment, the cyclodextrin is a β-cyclodextrin or a modified β-cyclodextrin.

β-cyclodextrin has the following structure:

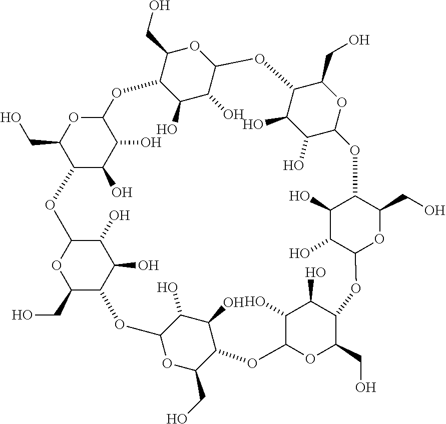

One or more of the hydroxyl groups of one or more of the sugar units may be modified, for example with an alkyl, alkenyl or alkynyl group, or with a substituted alkyl, alkenyl or alkynyl group. Therefore, in embodiments, the β-cyclodextrin may be unmodified or unsubstituted, or may be modified or substituted. As such, in a further embodiment, the β-cyclodextrin is a modified β-cyclodextrin. “Modified β-cyclodextrin” as used herein refers to a β-cyclodextrin that contains a modification at one or more hydroxyl groups of one or more sugar units of the β-cyclodextrin, i.e., a group or moiety that is attached to one or more hydroxyl groups of one or more sugar units of the β-cyclodextrin. As such, in embodiments, the modified β-cyclodextrin is an alkyl-, alkenyl-, alkynyl, substituted alkyl-, substituted alkenyl or substituted alkynyl-β-cyclodextrin (e.g., with a hydroxyl substitution). In embodiments, the alkyl, alkenyl or alkynyl groups are (C₁-C₆)alkyl, (C₁-C₆)alkenyl or (C₁-C₆)alkynyl groups. In a further embodiment, the modified β-cyclodextrin is a (C₁-C₆)alkyl β-cyclodextrin, in a further embodiment methyl-β-cyclodextrin (M-β-CD). In a further embodiment, the modified β-cyclodextrin is a hydroxy(C₁-C₆)alkyl β-cyclodextrin, in a further embodiment hydroxypropyl-β-cyclodextrin (HP-β-CD).

In an embodiment, the cyclodextrin is present in the pharmaceutical composition at a concentration of about 2 to about 15% (w/v), in a further embodiment about 2 to about 12.5% (w/v), for example about 2 to about 10% (w/v), about 2.5 to about 15% (w/v), about 2.5 to about 12.5% (w/v), about 2.5 to about 10% (w/v), about 5 to about 15% (w/v), about 5 to about 12.5% (w/v), about 5 to about 10% (w/v), about 7.5 to about 12.5% (w/v), about 7.5 to about 10% (w/v), about 5, 7.5, 10, 12.5 or 15% (w/v), or about 10% (w/v).

In an embodiment, the pharmaceutical composition of the present disclosure has a pH of about 4.5 to about 6.5, for example about 5.0 to about 6.0. According to another embodiment, the pharmaceutical composition has a pH of about 5.0. According to a further embodiment, the pharmaceutical composition has a pH of about 5.5. According to another further embodiment, the pharmaceutical composition has a pH of about 6.0.

In an embodiment, the pharmaceutical composition of the present disclosure comprises a diluent, for example an aqueous solution. In a further embodiment, the pharmaceutical composition comprises (typically sterile) water.

The pharmaceutical composition of the present disclosure may further contain other diluents, solubilizing agents, excipients, pH-modifiers, soothing agents, buffers, sulfur-containing reducing agents, antioxidants or the like, if desired. For example, sulfur-containing reducing agents include N-acetylcysteine, N-acetylhomocysteine, thioctic acid, thiodiglycol, thioethanolamine, thioglycerol, thiosorbitol, thioglycolic acid and salts thereof, sodium thiosulfate, glutathione, methionine and sulfhydryl-containing compounds such as thioalkanoic acid having 1 to 7 carbon atoms. Antioxidants include methionine, erythorbic acid, dibutylhydroxytoluene, butylhydroxyanisole, α-tocopherol, tocopherol acetate, L-ascorbic acid and salts thereof, L-ascorbyl palmitate, L-ascorbyl stearate, sodium bisulfite, sodium sulfite, triamyl gallate, propyl gallate or chelating agents such as disodium ethylenediamine tetraacetate (EDTA), sodium pyrophosphate, sodium metaphosphate. Other components commonly added may also be contained, e.g., inorganic salts such as sodium chloride, potassium chloride, calcium chloride, sodium phosphate, potassium phosphate, sodium bicarbonate; and organic salts such as sodium citrate, potassium citrate, sodium acetate.

In an embodiment, the pharmaceutical composition is stable at room temperature. A stable composition is a composition in which the active principal ingredient, i.e. the GHRH molecule (e.g., [trans-3-hexenoyl]hGHRH (1-44) amide) therein essentially retains its physical and chemical stability and integrity upon storage. Various analytical techniques for measuring protein or peptide stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the composition may be kept, for example, at 40° C. for 2 weeks to 1 month (and for up to 6 months), at which time stability is measured. The composition may also be kept, for example, at in ambient room temperature conditions (about 15-30° C., preferably about 20-25° C.) for at least 6 months, at which time stability is measured. The composition of the present disclosure preserves the stability of the GHRH molecule (e.g., [trans-3-hexenoyl]hGHRH (1-44) amide) in lyophilized form for a period of storage at room temperature (i.e. 20-25° C.) for at least 1 week, at least 2 weeks, at least 4 weeks, at least 6 weeks, at least 8 weeks, at least 3 months, at least 4 months, at least 6 months, or at least 12 months. For example, a “stable” composition may be one wherein more than about 80%, more than about 90%, more than about 95%, more than about 96%, more than about 97%, more than about 98%, or more than about 99% of the non-degraded active agent is present in the composition upon the storage period. The stability of the composition of the present disclosure may for example be measured using reverse phase high pressure liquid chromatography (RP-HPLC).

The pharmaceutical composition of the present disclosure may be useful for inducing or increasing GH secretion in a subject.

Accordingly, in another aspect, the present disclosure provides a method for inducing or increasing GH secretion in a subject in need thereof, said method comprising administering to said subject an effective amount of the above-mentioned formulation or composition.

In another aspect, the present disclosure provides a use of the above-mentioned formulation or composition, for inducing or increasing growth hormone secretion in a subject.

In another aspect, the present disclosure provides a use of the above-mentioned formulation or composition, for the preparation of a medicament for inducing or increasing GH secretion in a subject.

The terms “stimulating,” “increasing,” or “inducing” or any variations of these terms as used herein, refer to a measurable increase of a biological activity. In embodiments, the increase is at least a 10%, 20%, 40%, 60%, 80%, 90%, 95%, 100% (2-fold), 200% (3-fold) increase in the biological activity relative to a control. For example, a GRF analog is found to stimulate GHRHr activity when an increase in GH levels is measured following administration of the GHRH molecule to a subject (e.g., animal, human) in comparison to a subject not administered with the GHRH molecule.

In view of their GHRHr agonist activity and GH-releasing properties, the compositions of the disclosure may be useful as a medicament, for prophylactic and/or therapeutic applications in which stimulation of GH secretion is desirable, for example for the treatment or prevention of conditions/disorders/diseases associated with GHRH and/or GH function (e.g., in which reduced GH and/or GHRH function is involved in the etiology of the disease/disorder). Diseases and conditions in which administration of GH, GHRH or GHRH analogs/derivatives may be beneficial have been extensively described in the art (see, e.g., WO 2009/009727, WO 2006/042408, WO 2005/037307, WO 2004/105789). Such conditions/disorders/diseases include, for example, syndromes associated with fat accumulation, hypercholesterolemia, obesity, syndrome X, lipohypertrophy, lipoatrophy, lipodystrophy (e.g., HIV-associated lipodystrophy syndrome), impaired cognitive function, impaired daytime vigilance, declined function of the immune system (e.g., immunodeficiencies such as T-cell deficiencies), muscle protein catabolism, diseases/conditions associated with muscle wasting such as sarcopenia, frailty, radiotherapy- and/or chemotherapy-related side effects (e.g., in HIV-infected and cancer patients), cachexia (e.g., in cancer patients), hypothalamic pituitary dwarfism, burns, osteoporosis, renal failure, non-union bone fracture, acute/chronic debilitating illness or infection, wound healing, post-surgical problems, lactation failure, infertility in women, neurodegenerative conditions, GRF receptor-dependent tumors, conditions related to aging, sleep disorders/impairment, Non-Alcoholic Fatty Liver Disease (NAFLD) or Nonalcoholic steatohepatitis (NASH).

Therefore, in other aspects, the present disclosure provides a method for (1) stimulating daytime vigilance and/or cognitive function, e.g. in conditions related to aging, mild cognitive impairment (MCI), pre-Alzheimer's symptoms (Pre-Onset Alzheimer's), dementia and/or sleep impairment (e.g., age-related sleep impairment), (2) improving/preventing/treating metabolic conditions associated with fat accumulation and/or hypercholesterolemia (obesity, abdominal obesity/adiposity, abdominal obesity with metabolic disorders, abdominal obesity with relative GH deficiency, metabolic syndrome or syndrome X, lipohypertrophy, lipoatrophy, lipodystrophy (e.g., HIV-associated lipodystrophy syndrome), dyslipidemia, hypertriglyceridemia), NAFLD/NASH (3) improving anabolism in catabolic/wasting conditions, such as those observed in acute or chronic renal failure (e.g., acute or chronic renal failure wasting), chronic heart failure (e.g., chronic heart failure wasting), chronic obstructive pulmonary disease (COPD), cystic fibrosis (e.g., cystic fibrosis wasting in adults), frailty, burns, infections (sepsis), muscular dystrophy, congestive heart failure, neurodegenerative conditions (Alzheimer's, pre-Alzheimer's syndromes, amyotrophic lateral sclerosis (ALS), Acquired Immune Deficiency Syndrome (AIDS), protein malnutrition following long-term corticosteroid therapy, following non-union bone fracture, hip fracture, trauma, or major surgery (post-surgical problems), osteoporosis, long-term immobilization, cancer-related cachexia, sarcopenia (e.g., age-related sarcopenia), gastro-intestinal (GI) malabsorption (Short Bowel Syndrome (SBS), Crohn's disease) particularly in elderly subjects, for example to increase muscle mass and/or function, (4) improving immune function or reconstitution of immunodeficient states (e.g., T-cell immunodeficiencies) such as that associated aging, HIV infection/AIDS or following high-dose chemotherapy and/or radiotherapy (in HIV-infected and cancer patients), (5) altering a lipid parameter ((a) decreasing cholesterol; (b) decreasing non-HDL cholesterol; (c) decreasing triglycerides; and/or (d) decreasing the ratio of total cholesterol/HDL cholesterol); (6) altering a body composition parameter ((a) increasing lean body mass; (b) decreasing trunk fat; (c) decreasing visceral fat; (d) decreasing abdominal girth; (e) decreasing visceral adipose tissue (VAT); and/or (f) decreasing the VAT/subcutaneous adipose tissue (SAT) ratio), (7) enhancing fertility or treating infertility (in women), treating lactation failure, (8) treating GH deficiency (e.g., GH deficiency with abdominal obesity), providing GH replacement therapy, e.g., in adults, treating idiopathic short stature (ISS) (9) treating GHRH receptor-related tumors, (10) treating hypothalamic pituitary dwarfism, (11) improving wound healing, (12) treating burns, (13) treating acute/chronic debilitating illness or infection, and/or (14) preventing/treating a condition characterized by deficient or decreased bone formation (e.g., osteoporosis); the method comprising administering an effective amount of the above-mentioned composition, to a subject in need thereof.

In other aspects, the present disclosure provides a use of the above-mentioned composition for achieving one or more of the biological/therapeutic effects (1) to (14) noted above, e.g. for improving, preventing and/or treating the conditions, diseases or disorders noted above, or for the preparation/manufacture of a medicament for improving, preventing and/or treating the conditions, diseases or disorders noted above. In other aspects, the present disclosure provides the above-mentioned composition for use in improving, preventing and/or treating the conditions, diseases or disorders noted above, or for the preparation/manufacture of a medicament for improving, preventing and/or treating the conditions, diseases or disorders noted above.

The term “treatment” as used herein, is defined as the application or administration of a therapeutic agent to a subject, or application or administration of a therapeutic agent to an isolated tissue or cell line from a subject, who has a disorder, a disease, a symptom of disorder or disease, or a predisposition toward a disorder or disease, with the purpose to cure, heal, alleviate, delay, relieve, alter, remedy, ameliorate, improve or affect the disorder/disease, the symptoms of disorder/disease or the predisposition toward disorder/disease.

In another aspect, the present disclosure provides a method of administering a GHRH molecule to a subject, preferably trans-3-hexenoyl-GHRH₍₁₋₄₄₎-NH₂, to a subject to obtain plasmatic levels of the GHRH molecule that are bioequivalent to administration of 2 mg of the GHRH molecule at a concentration of 1 mg/mL (e.g., the EGRIFTA™ formulation comprising 5% mannitol), the method comprising administering to the subject about 1.3 to about 1.5 or 1.6 mg of the GHRH molecule at a concentration of about 3.5 mg/mL or more. In an embodiment, the GHRH molecule, preferably trans-3-hexenoyl-GHRH₍₁₋₄₄₎-NH₂, is formulated in the pharmaceutical composition described herein. In an embodiment, the subject suffers from one or more of the conditions, diseases or disorders noted above. In a further embodiment, the subject suffers from HIV-associated lipodystrophy.

The term “bioequivalent” as used herein means that one or more pharmacokinetic (PK) parameters following administration of the GHRH molecule to subjects do not significantly differ between the two treatment regimens, as determined using a suitable statistical standard. In an embodiment, at least two PK parameters do not significantly differ between the two treatment regimens. In an embodiment, at least three PK parameters do not significantly differ between the two treatment regimens. In an embodiment, the one or more PK parameters comprise the maximum plasmatic concentration (Cₘₐₓ). In an embodiment, the one or more PK parameters comprise the area under the plasma concentration time curve extrapolated to infinity (AUC_{0-∞}). In an embodiment, the one or more PK parameters comprise the cumulative area under the plasma concentration time curve calculated from 0 to T_{LQC} (time of last observed quantifiable plasma concentration) using the linear trapezoidal method (AUC_{0-T}). In an embodiment, the natural logarithmic (In) transformation of the one or more PK parameters is used for the statistical analysis. In an embodiment, the statistical standard used is the ratio of geometric LSmeans with corresponding 90% confidence interval (CI) for the exponential of the difference between the two treatment regimens for the Least-squares means (LSmeans) of the In-transformed PK parameter(s) that is within the 80.00% to 125.00% range, as described in the Examples below.

In an embodiment, the method permits to achieve a maximum plasmatic concentration (Cₘₐₓ) of the GHRH molecule of about 1500 to about 4500 pg/mL in a human subject. In another embodiment, the method permits to achieve an average Cₘₐₓ of the GHRH molecule of about 2500 to about 3500 pg/mL in a population of human subjects. In further embodiments, the method permits to achieve an average maximum plasmatic concentration Cₘₐₓ of the GHRH molecule of about 2600 or 2700 to about 3000, 3100 or 3200 pg/mL in a population of human subjects.

In an embodiment, the method permits to achieve an area under the plasma concentration time curve extrapolated to infinity (AUC_{0-∞}) of the GHRH molecule of about 300 to about 1400 p·gh/mL in a subject. In an embodiment, the method permits to achieve an average AUC_{0-∞} of the GHRH molecule of about 500 to about 1000 p·gh/mL in a population of human subjects. In further embodiments, the method permits to achieve an average AUC_{0-∞} of the GHRH molecule of about 600, 650 or 700 to about 750, 800, 850 or 900 pg/mL in a population of human subjects.

In an embodiment, the method comprises: (a) resuspending a lyophilized GHRH molecule, preferably trans-3-hexenoyl-GHRH₍₁₋₄₄₎-NH₂ or a pharmaceutically acceptable salt thereof, in a suitable volume of a pharmaceutically acceptable diluent to obtain a GHRH molecule solution at about 3.5 mg/mL or more; and (b) administering a suitable volume of the GHRH solution so that about 1.3 to about 1.6 or 1.5 mg of the GHRH molecule, preferably trans-3-hexenoyl-GHRH₍₁₋₄₄₎-NH₂ or a pharmaceutically acceptable salt thereof, is administered to the subject.

In an embodiment, the method comprises: (a) resuspending a lyophilized GHRH molecule, preferably trans-3-hexenoyl-GHRH₍₁₋₄₄₎-NH₂ or a pharmaceutically acceptable salt thereof, in a suitable volume of a pharmaceutically acceptable diluent to obtain a GHRH molecule solution at about 4 to about 8 mg/mL; and (b) administering about 0.16 to about 0.4 mL of the GHRH molecule solution of (a) to the subject, thereby administering about 1.3 to about 1.5 or 1.6 mg of the GHRH molecule.

In an embodiment, the method comprises: (a) resuspending a lyophilized GHRH molecule, preferably trans-3-hexenoyl-GHRH₍₁₋₄₄₎-NH₂ or a pharmaceutically acceptable salt thereof, in a suitable volume of a pharmaceutically acceptable diluent to obtain a GHRH molecule solution at about 3.8 to about 4.2 mg/mL; and (b) administering about 0.31 to about 0.4 mL of the GHRH molecule solution of (a) to the subject, thereby administering about 1.3 to about 1.5 or 1.6 mg of the GHRH molecule.

In a further embodiment, the method comprises: (a) resuspending lyophilized GHRH molecule, preferably trans-3-hexenoyl-GHRH₍₁₋₄₄₎-NH₂ or a pharmaceutically acceptable salt thereof, in a suitable volume of a pharmaceutically acceptable diluent to obtain a GHRH molecule solution at about 4 mg/mL; and (b) administering about 0.35 mL of the GHRH molecule solution of (a) to the subject, thereby administering about 1.4 mg of the GHRH molecule.

In a further embodiment, the method comprises: (a) resuspending about 2 mg of lyophilized GHRH molecule, preferably trans-3-hexenoyl-GHRH₍₁₋₄₄₎-NH₂ or a pharmaceutically acceptable salt thereof, in about 0.5 mL of a pharmaceutically acceptable diluent to obtain a GHRH molecule solution at about 4 mg/mL; and (b) administering about 0.35 mL of the GHRH molecule solution of (a) to the subject, thereby administering about 1.4 mg of the GHRH molecule.

In an embodiment, the lyophilized GHRH molecule, preferably trans-3-hexenoyl-GHRH₍₁₋₄₄₎-NH₂ or a pharmaceutically acceptable salt thereof, is in a container, preferably a sealed container, such as a vial. In an embodiment, the lyophilized GHRH molecule is resuspended using a syringe. In an embodiment, the GHRH molecule solution is administered by injection, e.g., subcutaneous injection.

As used herein, the term “subject” or “patient” are taken to mean a warm-blooded animal such as a mammal, for example, a cat, a dog, a mouse, a guinea pig, a horse, a bovine cow, a sheep or a human. In an embodiment, the subject is a mammal. In a further embodiment, the above-mentioned subject is a human.

In another aspect, the present disclosure also provides a kit comprising: (a) a first container comprising at least about 1.3 to about 1.5 mg of lyophilized GHRH molecule, preferably trans-3-hexenoyl-GHRH₍₁₋₄₄₎-NH₂ or a pharmaceutically acceptable salt thereof; (b) a second container comprising a pharmaceutically acceptable diluent; and (c) instructions for resuspending the lyophilized GHRH molecule, preferably trans-3-hexenoyl-GHRH₍₁₋₄₄₎-NH₂ or a pharmaceutically acceptable salt thereof, in the pharmaceutically acceptable diluent to obtain a GHRH molecule solution at about 3.5 mg/mL or more.

In an embodiment, the kit comprises: (a) a first container comprising about at least about 1.3 to about 1.5 mg of lyophilized GHRH molecule, preferably trans-3-hexenoyl-GHRH₍₁₋₄₄₎-NH₂ or a pharmaceutically acceptable salt thereof; (b) a second container comprising at least 0.35 mL of a pharmaceutically acceptable diluent; and (c) instructions for resuspending the lyophilized GHRH molecule, preferably trans-3-hexenoyl-GHRH₍₁₋₄₄₎-NH₂ or a pharmaceutically acceptable salt thereof, in the pharmaceutically acceptable diluent to obtain a GHRH molecule solution at about 3.8 to about 4.2 am/mL.

In an embodiment, the first container comprises about 2 mg of lyophilized trans-3-hexenoyl-GHRH₍₁₋₄₄₎-NH₂ or a pharmaceutically acceptable salt thereof. In an embodiment, the second container comprises about 0.5 mL of the pharmaceutically acceptable diluent.

In an embodiment, the pharmaceutically acceptable diluent is an aqueous solution, for example sterile water.

In an embodiment, the lyophilized GHRH molecule, preferably trans-3-hexenoyl-GHRH₍₁₋₄₄₎-NH₂ or a pharmaceutically acceptable salt thereof, is in a sealed container, such as a vial. In an embodiment, the kit further comprises at least one syringe.

In an embodiment, the kit further comprises instructions for administering about 1.3 to about 1.5 mg of the GHRH molecule to the subject, e.g., by subcutaneous injection.

### MODE(S) FOR CARRYING OUT THE INVENTION

The present invention is illustrated in further details by the following non-limiting examples.

### Example 1: PK Studies in Dogs—4 mg/mL Formulations

Materials and Methods.

The purpose of this analysis was to calculate the pharmacokinetic parameters for tesamorelin (TH9507) in stabilized dog plasma (K₂EDTA) following subcutaneous (SC) administration of 4 different formulations of TH9507 (Egrifta™, Formulation #4, Formulation #4′ pH 5.0 and Formulation #4′ pH 5.5) and to compare the bioavailability of TH9507 in the different formulations. Twelve male beagle dogs (7.0 to 8.6 kg, 5 to 7 months old at onset of treatment) were divided into 4 dose groups (2 mg/day, one dose of one formulation per week) in 4 alternative sequences in a PK study (Table 1).

TABLE 1
Study design

| | | | | | Dose level | Number of |
|---|---|---|---|---|---|---|
| Group # | Day 1 | Day 8 | Day 15 | Day 22 | (mg/day)* | animals |
| 1 | Egrifta ™ | Formulation | Formulation | Formulation | 2 | 3 |
| | | #4 | #4′ pH 5.0 | #4′ pH 5.5 | | |
| 2 | Formulation | Egrifta ™ | Formulation | Formulation | 2 | 3 |
| | #4′ pH 5.5 | | #4 | #4′ pH 5.0 | | |
| 3 | Formulation | Formulation | Egrifta ™ | Formulation | 2 | 3 |
| | #4′ pH 5.0 | #4′ pH 5.5 | | #4 | | |
| 4 | Formulation | Formulation | Formulation | Egrifta ™ | 2 | 3 |
| | #4 | #4′ pH 5.0 | #4′ pH 5.5 | | | |

*Egrifta ™ concentration, once reconstituted was 1 mg/mL, dose volume was 2 mL. Formulation #4, Formulation #4′ pH 5.0 and Formulation #4′ pH 5.5 concentration, once reconstituted was 4 mg/mL, dose volume was 0.5 mL. See below for a description of the contents of the different formulations.

The contents of the formulations tested were as follows:

TABLE 2
Formulations tested

| Formulation | | Reconstitution |
|---|---|---|
| name | Vehicle Component | volume |
| Egrifta ™ | 5% (w/v) mannitol | 2.2 mL |
| Formulation | 4% mannitol, 2% sucrose, | 0.6 mL |
| #4 | 10 mM Histidine, 0.01% Tween ™ | |
| Formulation | 4% mannitol 2% sucrose | 0.6 mL |
| #4’ pH 5.0 | | |
| Formulation | 4% mannitol 2% sucrose | 0.6 mL |
| #4’ pH 5.5 | | |

In order to verify the concentration of tesamorelin in the formulations, representative samples (0.25 mL in duplicate) were taken from each concentration on each day of dose preparation (except for Egrifta™), and stored at +4° C. nominal. One aliquot of each formulation was analyzed by HPLC-UV.

The formulations were administered once by subcutaneous injection on either Day 1, 8, 15 or 22 using a hypodermic needle attached to a syringe. The dose volume was 2 mL for the Egrifta™ group animals and 0.5 mL for the Formulation #4 groups of animals, thus a dose of 2 mg tesamorelin in each case.

Pharmacokinetics. Blood samples (2 mL) were collected by venipuncture into K₂EDTA tubes from all animals predose and at approximately 5, 10, 15, 20, 30, 40, 60, 80, 120 and 180 minutes following dosing. Blood samples were placed immediately on wet ice. Within 20 minutes of sample collection, blood samples were centrifuged at approximately 1000 g for 10 minutes in a refrigerated centrifuge. The resultant plasma was acidified by adding 50 μL of acidified Hanks' Balanced Salt Solution (supplied) to 450 μL of plasma and mixed by vortexing within approximately 15 minutes from the start of centrifugation. The acidified plasma sample was aliquoted into 2× ˜250 μL in uniquely labeled 0.5 mL polypropylene screwed cap tubes. Following the addition of the stabilizer, samples were left at room temperature for circa 10 minutes prior to freezing in a freezer at approximately −80° C. (i.e., maximum of 25 minutes from the start of centrifugation to stabilization and samples put on dry ice. Maximum time between blood sample collection and time on dry ice/time of plasma stabilization should not have exceeded 45 minutes.

Data Analysis.

The maximum observed concentration, Cₘₐₓ, was determined by inspection of the data. Non-compartmental exposure, AUC₍₀₋ₜₗₐₛₜ₎ of tesamorelin using the reported concentrations in plasma was calculated by the trapezoid rule (GraphPad Prism version 3.00 for Windows, GraphPad Software, San Diego Calif. USA.

The statistical analysis on the pharmacokinetic parameters, AUC₀₋ₜ and Cₘₐₓ was conducted by using SAS version 9.2. A mixed model analysis using the MIXED procedure in SAS was performed to compare PK parameters between treatments and across periods, on In-transformed AUC₀₋ₜ and Cₘₐₓ. The first-order carryover effect was tested by incorporating covariates for the residual effects in the model; if the first-order carryover effect was found not statistically significant (at a level of 5%), these covariates were to be removed from the model. Factors incorporated in the model were Sequence, Group, Sequence*Group, Period nested within Group, Treatment and Treatment*Group as fixed factors, Subject nested within Sequence*Group as a random factor and first-order carryover effect as covariates; the interaction Treatment*Group was removed from the statistical model if found to be not statistically significant. The ratios (A/D, B/D and C/D) and 90% geometric confidence interval (CI) for the ratios, based on least squares means (LSM) of the In-transformed data were calculated.

Results.

There was no mortality treatment related clinical signs or affects on body weight noted during the study. The administration of TH9507 to the beagle dogs by subcutaneous injection was well tolerated.

Stabilization of tesamorelin is important during plasma sample collection in order to obtain accurate concentration results. Due to technical oversight, many of the pharmacokinetic samples were not stabilized within the validated value of 15 minutes. The delay (>15 minutes and up to ˜80 minutes) in the time to stabilization of the dog plasma samples resulted in an underestimation of the analyte concentration. Data from previous stability studies showed a linear relationship between the decline in recovery of tesamorelin and time to stabilization. A correction factor was derived from this function and was applied to the measured concentrations obtained in this study for samples that were stabilized beyond the validated value of 15 minutes. This experimentally-derived correction is considered valid for the purpose of the present study to evaluate if Formulation #4 and Formulation #4′ were similar in terms of bioavailability.

A significant carryover effect was not observed for either the Cₘₐₓ (p=0.5286) or the AUC₍₀₋ₜ₎ (p=0.0632) in this study, however there was a significant period effect for both pharmacokinetic parameters. Based on an ANOVA model without a carryover effect, a statistically significant (p=0.0054) period effect was observed for the AUC₍₀₋ₜ₎ and particularly for the Cₘₐₓ (p=0.0002).

The ratios and 90% confidence intervals showed similar results for all three of the 4 mg/mL formulations; Formulation #4, Formulation #4 (pH 5.0) and Formulation #4 (pH 5.5). Notably, the relative bioavailability for all of the 4 mg/mL formulations was markedly greater than that following administration of the 1 mg/mL formulation (Egrifta™) with ratios ranging from 147 to 159 (Table 3), even though the same dose (2 mg) of tesamorelin was administered.

TABLE 3
Statistical results for AUC₍₀₋ₜ₎

| Comparison | Ratio | Lower Limit | Upper Limit |
|---|---|---|---|
| Formulation #4-5/Formulation #4 | 92.352 | 78.273 | 108.964 |
| Formulation #4-5.5/Formulation #4 | 97.996 | 83.056 | 115.623 |
| Formulation #4-5.5/Formulation #4-5 | 106.111 | 89.934 | 125.197 |
| Formulation #4/Egrifta ™ | 159.024 | 134.781 | 187.628 |
| Formulation #4-5/Egrifta ™ | 146.863 | 124.474 | 173.279 |
| Formulation #4-5.5/Egrifta ™ | 155.837 | 132.080 | 173.279 |

For the Cₘₐₓ, the ratios and 90% confidence intervals again indicated similar results for all three of the 4 mg/mL formulations. The Cₘₐₓ values were greater following administration of any of the three 4 mg/mL formulations relative to Egrifta™ with ratios that ranged from 170 to 180 (Table 4), again, even though the same dose (2 mg) of tesamorelin was administered.

TABLE 4
Statistical results for Cₘₐₓ

| Comparison | Ratio | Lower Limit | Upper Limit |
|---|---|---|---|
| Formulation #4-5/Formulation #4 | 94.346 | 77.145 | 115.382 |
| Formulation #4-5.5/Formulation #4 | 95.709 | 78.259 | 117.049 |
| Formulation #4-5.5/Formulation #4-5 | 101.444 | 82.949 | 124.063 |
| Formulation #4/Egrifta ™ | 180.421 | 147.527 | 220.650 |
| Formulation #4-5/Egrifta ™ | 170.220 | 139.186 | 208.174 |
| Formulation #4-5.5/Egrifta ™ | 172.679 | 141.197 | 211.181 |

While there was a lack of a carryover effect in this study, there was a period effect that was found to be statistically significant for both AUC₀₋ₜ and Cₘₐₓ. Nonetheless, based on these PK parameters, the relative bioavailability for the variants of the reference formulation #4 (Formulation #4 (pH 5.0) and Formulation #4 (pH 5.5)) was essentially the same as that of the reference formulation #4, and all of the 4 mg/mL formulations were more bioavailable than Egrifta™ (tesamorelin 1 mg/I) in the dog.

### Example 2: PK Studies in Dosis—8 mg/mL Formulation

Materials and Methods

The purpose of this analysis was to calculate the pharmacokinetic parameters for tesamorelin (TH9507) in stabilized dog plasma (K₂EDTA) following subcutaneous (SC) administration of 2 different formulations of TH9507 and to compare the bioavailability of TH9507 in the different formulations. The formulations used were the following:

Formulation #1: 1 mg/mL of TH9507 in 5% mannitol, pH=6 (adjusted with NaOH);

Formulation #8: 8 mg/mL of TH9507 in 10% (w/v) 2-hydroxypropyl-β-cyclodextrin, 3% (w/v) D-mannitol, pH=6.

Male beagle dogs were administered the formulations by subcutaneous injection on four occasions as shown in Table 5.

TABLE 5
Study design

| Group | | | Dose level | Number of |
|---|---|---|---|---|
| # | Day | Treatment | (mL/injection)* | animals |
| 1 | 1 | Formulation #1 (1 mg/mL) | 2 | 3 |
| | 4 | Formulation #8 (8 mg/mL) | 0.2 | |
| | 7 | Formulation #1 (1 mg/mL) | 2 | |
| | 10 | Formulation #8 (8 mg/mL) | 0.2 | |
| 2 | 1 | Formulation #8 (8 mg/mL) | 0.2 | 3 |
| | 4 | Formulation #1 (1 mg/mL) | 2 | |
| | 7 | Formulation #8 (8 mg/mL) | 0.2 | |
| | 10 | Formulation #1 (1 mg/mL) | 2 | |

*Formulation #1: 2 mL of 1 mg/mL TH9507 = 2 mg; Formulation #8: 0.2 mL of 8 mg/mL TH9507 = 1.6 mg.

A series of 10 blood samples (approximately 1 mL each) were collected from each dog on each of days 1, 4, 7 and 10. For this purpose, each dog was bled by venipuncture and the samples were collected into tubes containing K₂EDTA and 65 μL of AHBSS (Acidified Hank's Balanced Salt Solution). The samples were mixed and then placed on wet ice pending processing. The sampling was performed as follows: pre-dose (−15 and −5 minutes), 5, 10, 15, 30, 45, 60, 90 and 120 min. post-dose. The samples were centrifuged (approximately 4° C.) for 30 seconds in a microcentrifuge at top speed within 15 minutes of collection and the resulting plasma was frozen in dry-ice/isopropyl alcohol bath and stored at −80° C. until further analyses.

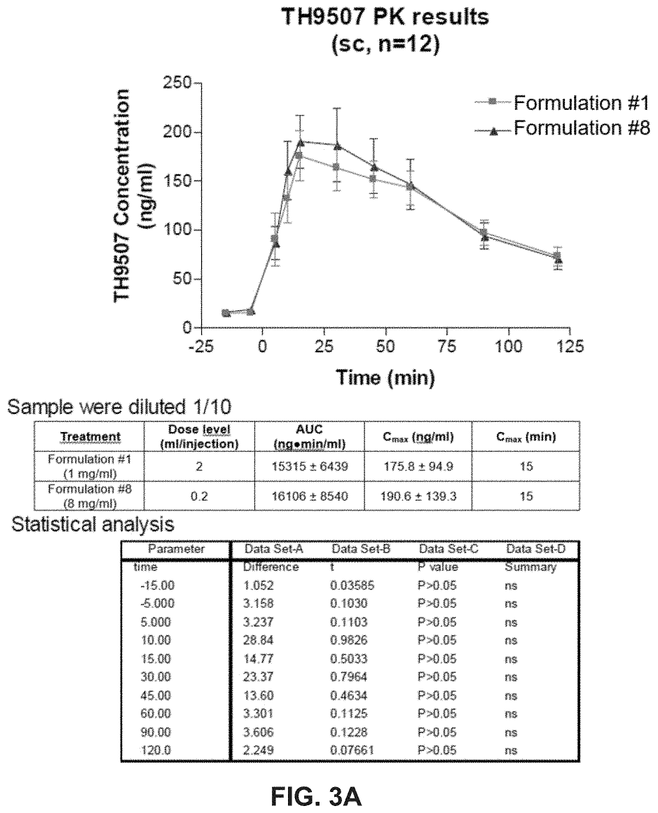

A competitive ELISA method was developed to measure TH9507 in dog plasma over the nominal range of 0.1-100 ng/mL. A rabbit affinity purified polyclonal antibody directed to the N-terminus of TH9507 was used as the capture antibody. The plasma levels of TH9507 were determined by the displacement of a bound analogue of TH9507 labelled at the C-terminus with biotin using a standard curve (FIG. 2).

Data Analysis.

The data were analyzed using one-way analysis of variance. Bonferroni's test was used to make comparisons between different treatments. These methods were available in Graphpad Prism software.

Results.

The PK profile of 1.6 mg of formulation #8 was compared to that of 2 mg of formulation #1 in a cross-over study in male beagle dogs. Formulations #1 and #8 doses were injected as shown above (Table 6) to two groups (n=3) dogs on four different days (1, 4, 7, 10). The plasma levels of TH9507 were determined using a competitive ELISA. The capture antibody was directed to the amino terminal portion of TH9507 and was shown not to cross-react with endogenous GRF. The bound biotinylated GHRH₍₁₋₄₄₎ was detected with streptavidin-HRP in chemiluminescent reaction.

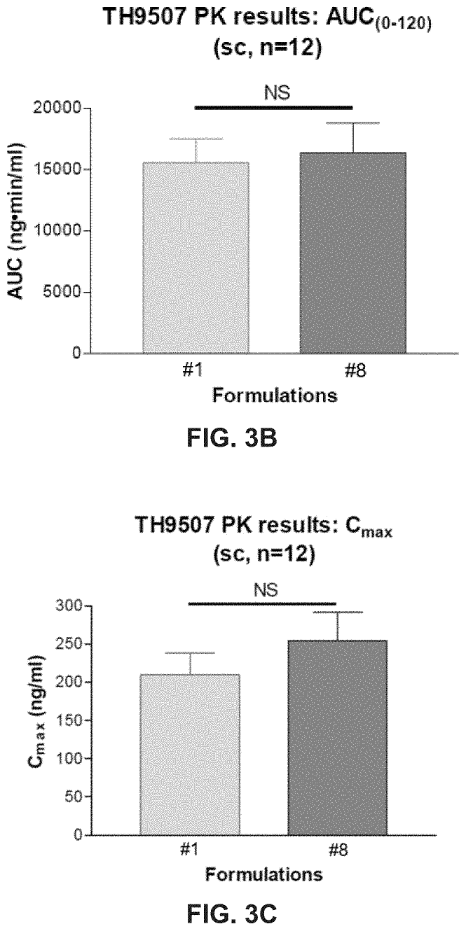

The average PK profiles of TH9507 obtained with formulations #1 and #8 were similar and the differences were not significant at any time point. The PK parameters, Tₘₐₓ, Cₘₐₓ and AUC₍₀₋₁₂₀₎ were similar and the differences were not significant between formulations #1 and #8 (FIGS. 3A-3C). Thus, it may be concluded that formulation #1 (2 mg) and formulation #8 (1.6 mg) produced pharmacokinetically equivalent profile of TH9507 in beagle dogs, and that formulation #8 has an increased bioavailability relative to formulation #1.

### Example 3: Bioequivalence Study in Humans with the 4 mg/ml Formulation

Materials and Methods

This was a single centre, pilot, bioequivalence, phase 1, open-label, randomised, 4-period, 4-way, crossover study to determine the relative bioavailability of tesamorelin's formulations after a single subcutaneous administration at different dosages to identify which dose of the 4 mg/mL formulation will be used in the bioequivalence study with the 1 mg/mL formulation (Egrifta™) and to evaluate the safety and tolerability of tesamorelin formulations. There were 4 treatments in this study, the 1 mg/mL formulation (Treatment D) and three dosages of the 4 mg/mL formulation (Treatments A, B, and C). A total of 32 healthy adult, naïve-tesamorelin subjects were enrolled and dosed in the study.

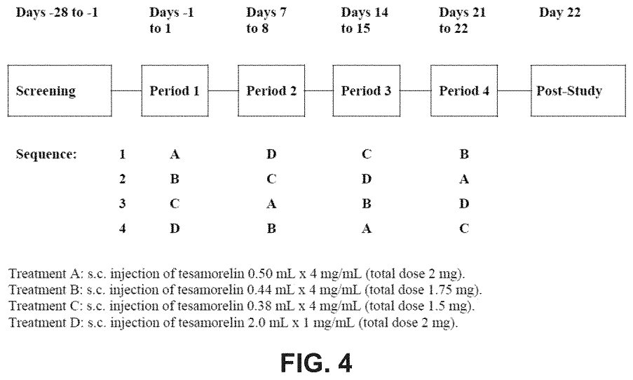

The study design is depicted in FIG. 4.

Subject Inclusion Criteria:
Male or female (a maximum of 50% female subjects was to be included), smoker or nonsmoker, 18 and 65 years of age.
Capable of consent.
Body mass index (BMI) ≥20.0 and <30.0 kg/m².

Subjects were administered a single subcutaneous dose of tesamorelin as a 0.50 mL×4 mg/mL (2 mg), 0.44 mL×4 mg/mL (1.75 mg), 0.38 mL×4 mg/mL (1.5 mg), or 2.0 mL×1 mg/mL (2 mg) injection solution in each period.

The following products were administered:

TABLE 6
Description of formulations studied

| | Study Drug (Treatments | Study Drug |
|---|---|---|
| Parameter | A, B, and C) | (Treatment D) |
| Product | tesamorelin | tesamorelin |
| Strength | 4 mg/mL | 1 mg/mL |
| Dosage Form | Lyophilised powder to be | Lyophilised powder to be |
| | reconstituted for injection | reconstituted for injection |
| | solution | solution |
| Dose | A: 2 mg (0.50 mL), | 2 mg (2.0 mL) |
| Administered | B: 1.75 mg (0.44 mL), or | |
| | C: 1.5 mg (0.38 mL) | |
| Route of | Subcutaneous injection | Subcutaneous injection |
| Administration | (abdomen) | (abdomen) |
| Inactive | 4% Mannitol, 0.01% | 5% Mannitol |
| Ingredient | polysorbate, 2% sucrose, | |
| | 0.16% histidine | |

For quantification of tesamorelin, all blood samples were drawn into blood collection tubes (1×6 mL) containing ethylenediaminetetraacetic (edetic) acid dipotassium salt (EDTA K₂) prior to study drug administration and 0.050, 0.100, 0.150, 0.200, 0.250, 0.333, 0.500, 0.667, 1.00, 1.33, 2.00, 3.00, and 4.00 hours post-dose in each period (Days 1, 8, 15, and 22).

Plasma concentrations after each administration period (on Days 1, 8, 15 and 22) were used to calculate the following parameters by standard non-compartmental methods for tesamorelin:
AUC₀₋ₜ: area under the concentration-time curve from time zero to the last non-zero concentration, calculated by the linear trapezoidal method;
AUC_{0-∞}: area under the concentration-time curve from time zero to infinity (extrapolated);
Cₘₐₓ: maximum observed concentration;
AUC_{t/inf}: the ratio of AUC₀₋ₜ on AUC₀₋ᵢₙₜ;
Tₘₐₓ; time of observed Cₘₐₓ;
T_{1/2 el}; time required for one half of the total amount of a particular substance in a biological system to be degraded by biological processes
CI/F: Dose/AUC₀₋ᵢₙₜ
V_{d}/F: Volume of distribution/AUC_{0-inf}
Kₑₗ: Elimination rate constant

The number of subjects (N), arithmetic mean, median (with IQR), min., max., SD and CV are presented per treatment group for the PK parameters listed above. Moreover, Geo. means and CV (derived from the In-transformed parameters) were presented for the PK parameters AUC₀₋ₜ, AUC_{0-inf}, and Cₘₐₓ.

An analysis of variance (ANOVA) was performed to compare PK parameters between treatments and across periods, using the MIXED procedure in SAS, on untransformed Tₘₐₓ, T_{1/2el} and Kₑₗ and on In-transformed AUC₀₋ₜ, AUC_{0-inf} and Cₘₐₓ at the α level of 0.05. Factors incorporated in the model were Sequence, Group, Sequence*Group, Period nested within Group, Treatment and Treatment*Group as fixed factors, Subject nested within Sequence*Group as a random factor and first-order carryover effect as covariates. Probability (p) values were derived from Type III sums of squares. The first-order carryover effect was tested by incorporating covariates for the residual effects in the model. A CONTRAST statement was used to perform a global F-test for the first-order carryover effect. If the first-order carryover effect was found not statistically significant (at a level of 5%), these covariates were removed from the model, otherwise they were left in the model. The interaction Treatment*Group was tested using a α level of 0.05 and removed from the model if not found statistically significant. If the interaction Treatment*Group was found statistically significant, the results were presented by group and combined. For the between treatment comparisons, the ratio of LSMs (A/D, B/D and C/D) and the corresponding 90% Geo. CI were obtained using the LSM statement with the PDIFF and CL options from the analysis of the In-transformed data for AUC₀₋ₜ, AUC_{0-inf} and Cₘₐₓ. The intra-subject CV was estimated. Non-parametric statistical analysis was also performed on Tₘₐₓ using the method of Hauschke.

Results

The difference in absorption between the test dosages and the reference formulation, based on the Test/Reference ratios, are summarized in Table 7.

TABLE 7
Difference in PK parameters between the 4 ma/mL dosaaes (Treatments
A, B and C) and the reference formulation #1 (Treatment D)

| Parameter | A vs. D | B vs. D | C vs. D |
|---|---|---|---|
| AUC₀₋ₜ | ↑32% | ↑20% | ↑4% |
| AUC_{0-inf} (FDA) | ↑28% | ↑17% | ↑1% |
| AUC_{0-inf} (HPFB) | ↑28% | ↑17% | ↑2% |
| Cₘₐₓ | ↑39% | ↑32% | ↑14% |

A = 0.50 mL × 4 mg/mL = 2 mg
B = 0.44 mL × 4 mg/mL = 1.75 mg
C = 0.38 mL × 4 mg/mL = 1.5 mg
D = 2.0 mL × 1 mg/mL = 2 mg
HPFB = Health Products and Food Branch's, Health Canada

The extent of absorption (AUCs) was approximately 30% and 20% greater following the administration of the 2 mg (0.50 mL×4 mg/mL (A)) and 1.75 mg (0.44 mL×4 mg/mL (B)) test dosages, respectively, when compared to the administration of a 2 mg dose of the Reference formulation (2.0 mL×1 mg/mL (D)). The increase in the rate of absorption following the administration of the 2 mg and 1.75 mg test dosages was even greater with a 39% and 32% increase in Cₘₐₓ, respectively, compared to the reference formulation.

A similar extent of absorption (AUCs) and a ˜14% greater rate of absorption (Cₘₐₓ) were obtained following the administration of the 1.5 mg test dosage (0.38 mL×4 mg/mL (C)) when compared to the reference formulation. For the C vs. D comparison, the 90% Geo. CIs were within or nearly comprised within the range of 80.00% to 125.00% for AUCs and Cₘₐₓ. Thus, when compared to 2 mg of reference formulation (1 mg/mL), the lowest dosage (1.5 mg) of test formulation (4 mg/mL) presented similar extent of absorption (AUCs) with a 90% C.I. within the range of 80.00% to 125.00% and a slightly greater rate of absorption (Cₘₐₓ) with a 90% C.I. which was nearly comprised within the range of 80.00% to 125.00% (102.77% to 126.65%). The 1.5 mg test dosage of the 4 mg/mL formulation exhibits a bioavailability that is similar to that of the 1 mg/mL formulation.

### Example 4: Bioequivalence Study in Humans with the 4 mg/mL Formulation (1.4 mg)

Materials and Methods

The study was a single-center, bioequivalence, phase 1, randomized, 2-period, 2-sequence, crossover design in healthy male and female subjects. The following products were administered under fasting conditions:

Test: Tesamorelin 4 mg/mL

Reference: EGRIFTA® 1 mg/mL as the free base (1.1 mg tesamorelin acetate, anhydrous)

TABLE 8
Test formulation (lyophilized form)

| | Quantity |
|---|---|
| Component | (mg/vial) |
| N-[trans-3-Hexenoyl]-Human | 2.0 |
| Growth Hormone-Releasing | |
| Factor (1-44) Acetate | |
| Mannitol USP | 20 |
| Sucrose NF | 10 |
| Histidine USP | 0.78 |
| Polysorbate 20 NF | 0.05 |

The lyophilized test formulation was reconstituted in 0.5 mL of sterile water for injection USP, and 0.35 mL was administered to the subject.

TABLE 9
Reference (EGRIFTA ®) formulation (lyophilized form)

| | Quantity |
|---|---|
| Component | (mg/vial) |
| N-[trans-3-Hexenoyl]-Human | 1.1 |
| Growth Hormone-Releasing | |
| Factor (1-44) Acetate | |
| Mannitol USP | 55 |

The content of two vials of the lyophilized reference formulation was reconstituted in 2.2 mL of sterile water for injection USP, and 2 mL was administered to the subject.

A single subcutaneous dose of one of the following 2 treatments was administered in each study period according to the randomization scheme:

Treatment-A: Tesamorelin 4 mg/mL: Single subcutaneous dose of 1.4 mg (0.35 mL) (Test).

Treatment-B: Tesamorelin 1 mg/mL: Single subcutaneous dose of 2 mg (2.0 mL) (Reference)

A total of 28 subjects were included in this study and, after randomization, 27 subjects (96%) received Treatment-A and all 28 subjects received Treatment-B. Two subjects withdrew consent from the study; 26 subjects (93%) completed the study.

Subject Inclusion Criteria:
Tesamorelin-naïve male or female subject
Aged of at least 18 years but not older than 55 years
BMI within 18.5 to 30.0 kg/m²
Normal clinical laboratory values
No clinically significant diseases
Informed consent signed
Used of an acceptable method of contraception

Subject Exclusion Criteria:
Pregnant women
Presence of scars or tattoos at the injection site
History of hypersensitivity to tesamorelin or mannitol
Alcohol and drug abuse
Used of any enzyme-modifying drugs
HIV positive

After a supervised overnight fast, a single 1.4 mg (Treatment-A, 0.35 mL) or 2 mg (Treatment-B, 2 mL) subcutaneous dose of the tesamorelin formulation was administered into the abdomen (below the belly button to the left or right) in the morning on day 1 and day 8. The wash-out between periods was of 7 calendar days.

Blood samples were collected prior to and up to 4.00 hours after drug administration in K₂EDTA Vacutainers. Samples were processed and stored under conditions that have been shown not to cause significant degradation of the analyte. Briefly, samples were centrifuged at 4° C. and at approximately 1000 g for 10 minutes. The plasma obtained was transferred in a polypropylene transfer tube. Thereafter, 1800 μL of generated plasma were transferred into a polypropylene tube containing 200 μL of stabilization solution (10% of final volume). The stabilized plasma samples were put immediately on dry ice and stored frozen at −80° C. until assayed.

Tesamorelin plasma levels were assessed using a validated ELISA assay. The lower limit of quantitation (LOQ) and upper limit of quantitation were 150 pg/mL and 6000 pg/mL, respectively.

The main PK parameters of interest for this study were:
Cₘₐₓ (Maximum observed plasma concentration),
AUC_{0-T} (cumulative area under the plasma concentration time curve calculated from 0 to Time of last observed quantifiable plasma concentration (T_{LQC}) using the linear trapezoidal method); and
AUC_{0-∞} (Area under the plasma concentration time curve extrapolated to infinity, calculated as AUC_{0-T}+C_{LQC}/λ_{Z}, where C_{LQC} is the measured concentration at time T_{LQc}, and λ_{Z} is the apparent elimination rate constant, estimated by linear regression of the terminal linear portion of the log concentration versus time curve)

Other parameters such as Tₘₐₓ (Time of maximum observed plasma concentration; if it occurs at more than one time point, Tₘₐₓ is defined as the first time point with this value), AUC_{0-T∞} (Relative percentage of AUC_{0-T} with respect to AUC_{0-∞}), λ_{z} and T_{half} (Terminal elimination half-life, calculated as In(2)/λ_{Z}) were also determined.

The main absorption and disposition parameters were estimated using a non-compartmental approach with a log-linear terminal phase assumption. The trapezoidal rule was used to estimate the AUC (linear trapezoidal linear interpolation) and the terminal phase was estimated by maximizing the coefficient of determination estimated from the log-linear regression model. However, disposition parameters were not estimated for individual concentration-time profiles where the terminal log-linear phase could be reliably characterized using the following criteria:
Phoenix® WinNonlin® Best fit range selection: R² of at least 80%
The corresponding terminal half-life value was lower than or equal to 2 times the time interval over which λ_{Z} was estimated (i.e. T_{half}≤twice the time interval difference between T_{LQC} and T_{LIN}).

Descriptive statistics were calculated for plasma concentrations at each individual time point and for all PK parameters. The individual plasma concentration/time profiles were presented using the actual sampling times whereas the mean plasma concentration/time profiles were presented using the theoretical sampling times.

The natural logarithmic transformation of Cₘₐₓ, AUC_{0-T} and AUC_{0-∞} was used for all statistical inference. The parameter Tₘₐₓ was analyzed using a non-parametric approach. Test of fixed period, sequence and treatment effects was based on the Wilcoxon's rank sum test (Mann-Whitney U-test). All other PK parameters were statistically analyzed using an Analysis of Variance (ANOVA) model.

Bioequivalence demonstration was based on the 4 mg/mL to 1 mg/mL ratio of geometric Least-Square means (LSmeans) with corresponding 90% CI for Cₘₐₓ, AUC_{0-T} and AUC_{0-∞} being within the 80% to 125% acceptance range. The 90% confidence interval (CI) for the exponential of the difference in LSmeans between the Test and Reference products was calculated for the In-transformed parameters (Treatment-A to Treatment-B ratio of geometric LSmeans).

The formula to estimate the intra-subject CV was: \sqrt{e^{MSE}−1}, where MSE is the Mean Square Error obtained from the ANOVA model of the In-transformed parameters.

Safety was assessed by qualified study staff by evaluating the following: reported adverse events (AEs), clinical laboratory test results, vital signs measurements, ECG findings, physical examination findings, visual skin evaluation and glycemia.

Results

Previous clinical studies have shown that the absolute bioavailability of tesamorelin after subcutaneous administration of a 2 mg dose was determined to be less than 4% in healthy adult subjects (EGRIFTA®, Product monograph). Single and multiple dose pharmacokinetics (PK) of tesamorelin have been characterized in healthy subjects and HIV-infected patients without lipodystrophy following 2 mg subcutaneous administration. The mean values and coefficient of variation (CV) of the extent of absorption for tesamorelin were 634.6 (72.4) pg·h/mL in healthy subjects, after a single subcutaneous administration of a 2 mg tesamorelin dose. The mean and CV peak tesamorelin concentration values were 2874.6 (43.9) pg/mL in healthy subjects. The median peak plasma tesamorelin concentration was 0.15 h. Based on this data from previous studies, the expected PK parameters observed after a single 2 mg subcutaneous dose of tesamorelin are the following (Table 10):

TABLE 10
Expected PK Parameters

| PK parameters | Tesamorelin |
|---|---|
| Cₘₐₓ (pg/mL) | 2874.6 (Inter-CV 43.9%) |
| Tₘₐₓ (hours)* | 0.15 |
| T_{half} (hours) | 0.4 |

*For Tₘₐₓ, median is presented

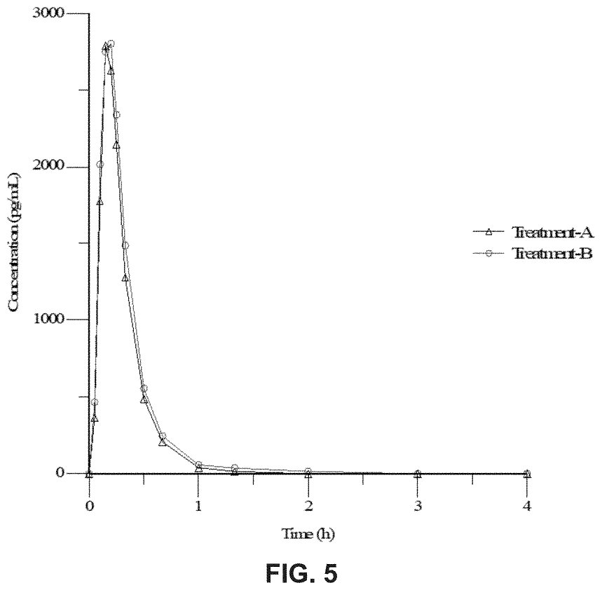

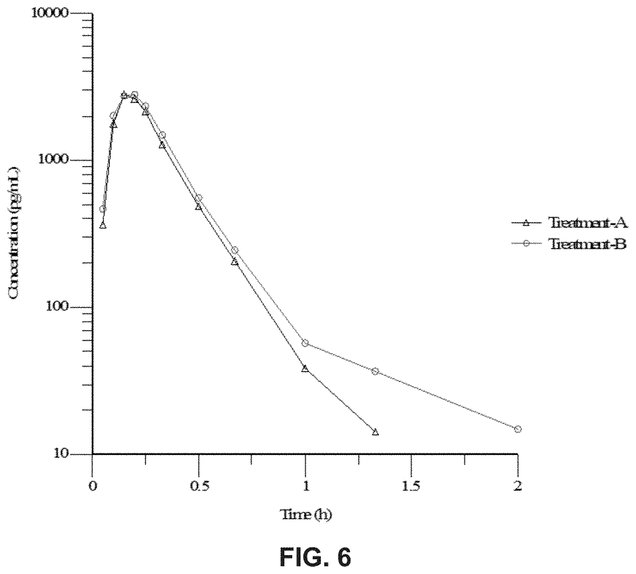

In the present study, several PK parameters were measured and compared in subjects administered with a single 1.4 mg (Treatment-A, 0.35 mL) or 2 mg (Treatment-B, 2 mL) subcutaneous dose of tesamorelin. Mean plasma concentration-time profiles are displayed by treatment in FIG. 5 (linear scale) and FIG. 6 (logarithmic scale). Plasma levels were below the LOQ (150 pg/mL) in all samples collected prior to dosing. The wash-out period between doses was considered appropriate.

PK parameter values by treatment (n=26) are presented in Table 11. The terminal phase of tesamorelin could not be adequately estimated for 2 subjects. For these subjects, the criteria predefined in the protocol were not met, and the parameters λ_{Z}, AUC_{0-∞}, AUC_{0-T/∞} and T_{half} were not calculated for these periods; therefore, 23 observations were included in the statistical analysis.

TABLE 11
Summary of Plasma Tesamorelin PK Parameters

| | TREATMENT-A | | TREATMENT-B | |
|---|---|---|---|---|
| | (n = 26)^{b} | | (n = 26)^{b} | |
| PARAMETER | MEAN | CV (%) | MEAN | CV (%) |
| Cₘₐₓ (pg/mL) | 2956.1 | (47.4) | 3020.9 | (40.6) |
| In (Cₘₐₓ) | 7.8859 | (6.0) | 7.9329 | (5.2) |
| Tₘₐₓ (hours)^{a} | 0.15 | (0.15-0.25) | 0.20 | (0.15-0.33) |
| AUC_{0-T} (pg · h/mL) | 809.4 | (60.1) | 914.4 | (53.3) |
| In (AUC_{0-T}) | 6.5566 | (8.1) | 6.6936 | (7.6) |
| AUC_{0-∞} (pg · h/mL) | 889.1 | (56.5) | 1011.8 | (52.6) |
| In (AUC_{0-∞}) | 6.6661 | (7.5) | 6.7943 | (7.5) |
| AUC_{0-T/∞} (%) | 93.71 | (4.0) | 93.27 | (5.6) |
| λ_{z} (hours⁻¹) | 5.8217 | (30.9) | 5.5016 | (38.5) |
| T_{half} (hours) | 0.14 | (50.5) | 0.22 | (144.9) |
| Cl_{TOT}/F normalized by weight | 26.7 | (38.9) | 33.9 | (40.7) |
| ([L/h]/kg) | | | | |
| V_{D}/F normalized by weight(L/kg) | 4.8 | (39.4) | 8.0 | (90.9) |

^{a}Median and range are presented
^{b}n = 25 for AUC_{0-T} and n = 23 for AUC_{0-∞}, AUC_{0-T/∞}, λ_{z}, T_{half}, Cl_{TOT}/F normalized by weight and V_{D}/F normalized by weight

A summary of the statistical analysis of Cₘₐₓ, AUC_{0-T} and AUC_{0-∞} for tesamorelin is given in Table 12.

TABLE 12
Summary of the Statistical Analysis of Tesamorelin

| | INTRA- | | | | 90% CONFIDENCE | |
|---|---|---|---|---|---|---|
| | SUBJECT | GEOMETRIC LSMEANS^{a} | | RATIO | LIMITS (%) | |
| PARAMETER | CV (%) | TREATMENT-A^{b} | TREATMENT-B^{b} | (%) | LOWER | UPPER |
| Cₘₐₓ | 20.3 | 2659.6 | 2787.6 | 95.41 | 86.75 | 104.93 |
| AUC_{0-T} | 17.2 | 700.9 | 804.1 | 87.16 | 80.24 | 94.68 |
| AUC_{0-∞} | 17.9 | 790.2 | 897.3 | 88.06 | 80.49 | 96.36 |

^{a}units are pg/mL for Cₘₐₓ and pg · h/mL for AUC_{0-T} and AUC_{0-∞}
^{b}n = 26 for Cₘₐₓ, n = 25 for AUC_{0-T} and n = 23 AUC_{0-∞}

Information on adverse events (AEs) was collected during the study and continued through a safety follow-up visit. All AEs were classified according to MedDRA (version 20.1).

Twenty-two subjects (79%) reported a total of 72 AEs over the course of the study. Of these events, 10 were pre-treatment AEs and the remaining 62 were treatment emergent adverse events (TEAEs) experienced by 21 subjects (75%). The incidence of TEAEs was 52% and 57% in subjects dosed with 4 mg/mL and 1 mg/mL formulations, respectively.

The TEAEs experienced during the study were deemed mild (55/62; 89%) and moderate (7/62; 11%) in intensity. None of the subjects experienced a severe TEAE during the study. No SAEs and no deaths were reported in any of the subjects enrolled in this study. No subject was withdrawn by the investigator for safety reasons.

The PK data demonstrated that the geometric ratios of LSmeans for Cₘₐₓ, AUC_{0-T} and AUC_{0-∞} were 95.41%, 87.16% and 88.06%, respectively. For all parameters, the corresponding 90% CIs were included within the range of 80% to 125% defined as the bioequivalence acceptance range. A similar proportion of patients reported TEAEs related to the drug administration with both formulations (4 and 1 mg/mL).

A dose of tesamorelin 1.4 mg (0.35 mL) of a 4 mg/mL formulation is judged to be bioequivalent to a dose of 2 mg (2 mL) of the 1 mg/mL formulation and was found to be safe and well tolerated in healthy subjects.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1.-18. (canceled)

19. A method of administering trans-3-hexenoyl-GHRH₍₁₋₄₄₎-NH₂ or a pharmaceutically acceptable salt thereof to a subject to obtain plasmatic levels of trans-3-hexenoyl-GHRH₍₁₋₄₄₎-NH₂ or a pharmaceutically acceptable salt thereof that are bioequivalent to administration of 2 mg of trans-3-hexenoyl-GHRH₍₁₋₄₄₎-NH₂ at a concentration of 1 mg/mL, the method comprising administering to the subject a pharmaceutical composition comprising:
1.3 to 1.5 mg of trans-3-hexenoyl-GHRH₍₁₋₄₄₎-NH₂ or a pharmaceutically acceptable salt thereof at a concentration of 3.5 mg/mL to 4.5 mg/mL,
2% to 8% (w/v) of mannitol, and
0.1% to 5% (w/v) of sucrose,
wherein the pharmaceutical composition is free of cyclodextrin.

20. The method of claim 19, wherein the pharmaceutical composition comprises 1.38 to 1.42 mg of trans-3-hexenoyl-GHRH₍₁₋₄₄₎-NH₂ or a pharmaceutically acceptable salt thereof.

21. The method of claim 19, wherein the trans-3-hexenoyl-GHRH₍₁₋₄₄₎-NH₂ or pharmaceutically acceptable salt thereof is at a concentration of 3.8 to 4.2 mg/mL.

22. The method of claim 19, wherein the pharmaceutically acceptable salt of trans-3-hexenoyl-GHRH₍₁₋₄₄₎-NH₂ is an acetate salt.

23. The method of claim 19, wherein the pharmaceutical composition is administered by subcutaneous injection.

24. The method of claim 19, further comprising
resuspending lyophilized trans-3-hexenoyl-GHRH₍₁₋₄₄₎-NH₂ or pharmaceutically acceptable salt thereof in a suitable amount of a pharmaceutically acceptable diluent to obtain the pharmaceutical composition.

25.-30. (canceled)

31. The method of claim 19, wherein the pharmaceutical composition comprises 1.4 mg of trans-3-hexenoyl-GHRH₍₁₋₄₄₎-NH₂ or a pharmaceutically acceptable salt thereof.

32. The method of claim 19, wherein the trans-3-hexenoyl-GHRH₍₁₋₄₄₎-NH₂ or pharmaceutically acceptable salt thereof is at a concentration of 4 mg/mL.

33. The method of claim 19, wherein the pharmaceutical composition comprises 3% to 5% (w/v) of mannitol.

34. The method of claim 33, wherein the pharmaceutical composition comprises 4% (w/v) of mannitol.

35. The method of claim 19, wherein the pharmaceutical composition comprises 1% to 3% (w/v) of sucrose.

36. The method of claim 35, wherein the pharmaceutical composition comprises 2% (w/v) of sucrose.

37. The method of claim 19, wherein the pharmaceutical composition further comprises 0.001% to 1% (w/v) of surfactant.

38. The method of claim 19, wherein the pharmaceutical composition further comprises 0.01% (w/v) of surfactant.

39. The method of claim 37, wherein the surfactant is polysorbate-20.

40. The method of claim 19, wherein the pharmaceutical composition further comprises 0.05% to 0.5% (w/v) of histidine buffer.

41. The method of claim 40, wherein the pharmaceutical composition comprises 0.1% to 0.3% (w/v) of histidine buffer.

42. The method of claim 19, wherein the pharmaceutical composition has a pH of 5.0 to 6.0.

43. The method of claim 19, wherein the subject suffers from HIV-associated lipodystrophy.
